(19) United States
(12) Patent Application Publication
(10) Pub. No.: US 2020/0405715 A1
(43) Pub. Date: Dec. 31, 2020

(54) COMPOSITIONS AND METHODS FOR TREATING CANCER AND INFLAMMATORY DISEASES

(71) Applicant: University of Pittsburgh - Of the Commonwealth System of Higher Education, Pittsburgh, PA (US)

(72) Inventors: Beibei Chen, Wexford, PA (US); Yuan Liu, Wexford, PA (US)

(73) Assignee: University of Pittsburgh - Of the Commonwealth System of Higher Education, Pittsburgh, PA (US)

(21) Appl. No.: 16/078,353

(22) PCT Filed: Feb. 24, 2017
(86) PCT No.: PCT/US2017/019305

§ 371 Date: Aug. 21, 2018

Related U.S. Application Data
(60) Provisional application No. 62/300,451, filed on Feb. 26, 2016.
(60) Provisional application No. 62/325,842, filed on Apr. 21, 2016.

(51) Int. Cl. A61K 31/505; A61K 45/06; C07C 225/20; C07D 317/58; C07D 307/52; C07D 239/26; C07D 209/86; C07D 239/42; C07D 235/14; C07D 211/14; C07D 211/26; C07D 295/135; A61P 35/00; A61K 31/135; A61K 31/36; A61K 31/341; A61K 31/4468; A61K 31/4184; A61K 31/404; A61K 31/5375; A61K 31/343

(52) U.S. Cl. CPC A61K 31/505; A61K 31/343; C07C 225/20; C07D 317/58; C07D 307/52; C07D 239/26; C07D 209/86; C07D 239/42; C07D 235/14; C07D 211/14; C07D 211/26; C07D 295/135; A61P 35/00; A61K 31/135; A61K 31/36; A61K 31/341; A61K 31/4468; A61K 31/4184; A61K 31/404; A61K 31/5375; A61K 45/06

(57) ABSTRACT

The present disclosure is directed to, e.g., 3-substituted, 5-amine-substituted-cyclohex-2-en-1-one compounds, and method of treatment and making associated with the same compounds.

This application claims the benefit to priority from U.S. Provisional Patent Application No. 62/300,451, filed Feb. 26, 2016 and U.S. Provisional Patent Application No. 62/325,842, filed Apr. 21, 2016. All of the references listed above are incorporated herein by reference in their entirety.

### STATEMENT OF GOVERNMENT SUPPORT

This invention was made with government support under HL116472 awarded by the NIH. The government has certain rights in the invention.

### BACKGROUND

Dysregulated behavior of cellular protein degradation by ubiquitin E3 ligases and the downstream proteasome system is an emerging theme in cancer. Currently, several inhibitors were developed to focus on the proteasome system and proven to be effective in treating several human cancers. However, few researchers have been focused on targeting upstream ubiquitin E3 ligases. Cullin-RING ubiquitin ligases (CRLs) comprise the largest known category of ubiquitin ligases. Among all E3 ligases, Cullin-based complex E3 ligase such as SCF (also known as Cul1 and Skp1-Cullin-F-box), has profound roles in regulating cell cycle progression, mitosis, and DNA repair, all link to cancer formation and progression. Interestingly, the activity of SCF complex is highly dependent on NEDD8 conjugation of the Cullin protein. So far, there is only one inhibitor, MLN4924, which targets the upstream pathway of Cullin Neddylation, thus disrupting SCF E3 ligase-mediated protein ubiquitination and leading to cell cycle arrest and tumor cell apoptosis. However, several other downstream proteins are also involved in Cullin Neddylation, and protein DCN1 is one of the essential components of the NEDD8 conjugation pathway. DCN1 specifically promotes Cullin Neddylation, and enhances the activity of all Cullin-based complex E3 ligases.

Protein ubiquitination is the major protein processing function in cells. Ubiquitin (Ub) flags a targeted protein for degradation through the 26s proteasome or lysosome. Tanaka et al., “c-Cbl-dependent monoubiquitination and lysosomal degradation of gp130,” Mol. Cell Biol., 28:4805-18 (2008). Ubiquitin is conjugated to a target protein in a three-step process. First, an E1 ubiquitin-activating enzyme binds to ubiquitin via a thioester covalent bond. Then, the E1 transfers the ubiquitin to an E2 ubiquitin-conjugating enzyme. Finally, the C-terminus of Ub is attached to the ε-amino lysine (K) residue of the substrate, mediated by an ubiquitin E3 ligase. There are several families of these ubiquitin E3 ligases that include over 1,000 proteins (Jin et al., “Dual E1 activation systems for ubiquitin differentially regulate E2 enzyme charging,” Nature, 447:1135-8 (2007), Hatakeyama et al., “U box proteins as a new family of ubiquitin-protein ligases,” J. Biol. Chem., 276:33111-20 (2001)), of which the Skp-Cullin-F box (SCF) ubiquitin E3 ligase machinery are essential in regulating cell cycle progression, centrosome stability, and mitotic fidelity. Tyers et al., “One ring to rule a superfamily of E3 ubiquitin ligases,” Science, 284:601, 3-4 (1999); Guardavaccaro et al., “Control of chromosome stability by the beta-TrCP-REST-Mad2 axis,” Nature, 452:365-9 (2008); Seki et al., “Plkl- and beta-TrCP-dependent degradation of Bora controls mitotic progression,” J. Cell Biol., 181:65-78 (2008); Gusti et al., “The Arabidopsis thaliana F-box protein FBL17 is essential for progression through the second mitosis during pollen development,” PLoS One, 4:e4780 (2009); D'Angiolella et al., “SCF(Cyclin F) controls centrosome homeostasis and mitotic fidelity through CP110 degradation,” Nature, 466:138-42 (2010). The SCF complex contains a catalytic core consisting of Skp1, Cullin1, and the E2 ubiquitin-conjugating (Ubc) enzyme and an F box component that acts as a receptor targeting numerous substrates via phosphodegron elicited interactions. Zheng et al., “Structure of the Cul1-Rbx1-Skp1-F boxSkp2 SCF ubiquitin ligase complex,” Nature, 416:703-9 (2002); Cardozo et al., “The SCF ubiquitin ligase: insights into a molecular machine,” Nat. Rev. Mol. Cell Biol., 5:739-51 (2004); Cenciarelli et al., “Identification of a family of human F-box proteins,” Curr. Biol., 9:1177-9 (1999); Ilyin et al., “Identification of a novel Skp2-like mammalian protein containing F-box and leucine-rich repeats,” FEBS Lett., 459:75-9 (1999). Interestingly, SCF E3 ligase activity is heavily reliant upon Cullin protein modification by NEDD8, a process essential to SCF function. Morimoto et al., “Nedd8-modification of Cul1 is promoted by Roc as a Nedd8-E3 ligase and regulates its stability,” Biochemical and biophysical research Commc'ns, 301:392-8 (2003); Amir et al., “The NEDD8 pathway is essential for SCF(beta-TrCP)-mediated ubiquitination and processing of the NF-kappa B precursor p105,” J. of biological chemistry, 277:23253-9 (2002); Wu et al., “Conjugation of Nedd8 to CUL1 enhances the ability of the ROC1-CUL1 complex to promote ubiquitin polymerization,” J. of biological chemistry, 275:32317-24 (2000); Read et al., “Nedd8 modification of cul-1 activates SCF(beta(TrCP))-dependent ubiquitination of IkappaBalpha,” Molecular and cellular biology, 20:2326-33 (2000). NEDD8 pathway is homologous to ubiquitination pathway since NEDD8 is an ubiquitin like protein (UBL). Soucy et al., “An inhibitor of NEDD8-activating enzyme as a new approach to treat cancer,” Nature, 458:732-6 (2009). Similar to protein ubiquitination, Neddylation also occurs in a three step process. NEDD8 is first activated by an E1 enzyme (NEDD8 activating enzyme, NAE), then transferred to an E2 enzyme (Ubc12), before conjugated to target proteins. Id., Soucy et al. Since Neddylation of Cullin protein is essential for SCF function, previous efforts were focused on targeting Cullin1 Neddylation pathway as a new approach to treat cancer. So far, there is only one inhibitor, MLN4924, which targets the upstream pathway of Cullin Neddylation, the NAE protein, thus disrupting SCF E3 ligase-mediated protein ubiquitination and leading to cell cycle arrest and tumor cell apoptosis. However, several other downstream proteins are also involved in Cullin Neddylation, and specifically protein DCN1 is one of the essential components of the NEDD8 conjugation pathway. DCN1 functions by interacting with n-terminal of UBC12 E2 enzyme, which significantly facilitates and enhances NEDD8 conjugation to Cullin proteins. Thus DCN1 promotes Cullin Neddylation, and enhances the activity of all Cullin based complex E3 ligases. DCN1 protein overexpression promotes cellular growth, whereas DCN1 protein knockdown drastically inhibits cell proliferation. Broderick et al., “SCCRO promotes glioma formation and malignant progression in mice,” Neoplasia, 12:476-84 (2010); Fu et al., “Squamous Cell Carcinoma Related Oncogene (SCCRO) Family Members Regulate Cell Growth and Proliferation through Their Cooperative and Antagonistic Effects on Cullin Neddylation,” J. of Biol. Chem. (2016). DCN1 protein is also called SCCRO (squamous cell carcinoma-related oncogene), and heavily overexpressed in many types of cancers, which makes it an ideal target for cancer drug development. Fu et al. (2016); Huang et al., “The ubiquitin-associated (UBA) domain of SCCRO/DCUN1D1 protein serves as a feedback regulator of biochemical and oncogenic activity,” J. of Biol. Chem., 290:296-309 (2015).

There remains a need in the art for novel compounds and novel treatments to inhibit DCN1, which can greatly reduce Cullin Neddylation. There remains a need in the art for novel compounds and novel treatments to suppress the growth of human tumor cells.

There remains a need in the art for novel compounds and novel treatments to provide anti-inflammatory activity in vitro or in vivo and in murine lung and colon inflammation. Since SCF activity is required for NF-kB activation, DCN1 inhibitors exhibit excellent anti-inflammatory activity in vitro and in murine lung and colon inflammation models.

The present invention satisfies these needs. DCN1 inhibitors as described herein, therefore, hold promise for the treatment of cancer and various inflammatory disorders.

### SUMMARY OF THE DISCLOSED SUBJECT MATTER

The present invention is directed to novel compounds and novel treatments to inhibit DCN1, and to treat ailments and/or diseases. Examples of ailments and diseases that can be treated with the compounds and methods of the invention include, but are not limited to, various cancers, acute and chronic inflammation disorders such as asthma, chronic obstructive lung disease, pulmonary fibrosis, pneumonitis (including hypersensitivity pneumonitis and radiation pneumonitis), pneumonia, cystic fibrosis, psoriasis, arthritis/rheumatoid arthritis, rhinitis, pharyngitis, cystitis, prostatitis, dermatitis, allergy including hay fever, nephritis, conjunctivitis, encephalitis, meningitis, opthalmitis, uveitis, pleuritis, pericarditis, myocarditis, atherosclerosis, human immunodeficiency virus related inflammation, diabetes, osteoarthritis, psoriatic arthritis, inflammatory bowel disease (Crohn's disease, ulcerative colitis)/colitis, sepsis, vasculitis, bursitis, connective tissue disease, autoimmune diseases such as systemic lupus erythematosis (SLE), polymyalgia rheumatica, scleroderma, Wegener's granulomatosis, temporal arteritis, vasculitis, cryoglobulinemia, and multiple sclerosis, viral or influenza-induced inflammation, or edema.

Some embodiments of the disclosure include a compound represented by formula (I):

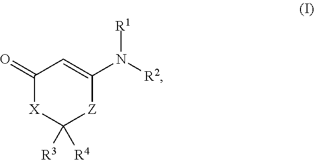

or a pharmaceutically acceptable salt, ester, hydrate thereof, wherein:
X and Z are independently C₀₋₂-alkyl, —(CH₂)ₛ—NH—(CH₂)ₜ—, —(CH₂)ₛ—O—(CH₂)ₜ—, or —(CH₂)ₛ—C(NH₂)—(CH₂)ₜ—, wherein s and t are independently an integer of 0 or 1;
R¹ is selected from the group consisting of H, optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl;
R² is selected from the group consisting of optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl;
R³ is selected from the group consisting of H, optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl; and
R⁴ is selected from the group consisting of optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl,
with the proviso that when X and Z are CH₂, and R¹ is H, R² is not an unsubstituted C₁-C₈ alkyl or an unsubstituted cyclohexyl or an unsubstituted C₁-C₂ alkyl-phenyl.

In some embodiments, the compound is selected from a compound of formula (I) or a pharmaceutically acceptable salt, ester, hydrate or prodrug thereof. In some embodiments, the compound is selected from a pharmaceutically acceptable salt of compound of formula (I). In some embodiments, R² is selected from the group consisting of C₁₋₁₆ alkyl, C₁₋₁₆ alkenyl, and C₁₋₁₆ alkynyl. In some embodiments, R² is selected from the group consisting of C₃₋₁₂ cycloalkyl, C₃₋₁₂ cycloalkyl-C₁₋₆alkyl, C₃₋₁₂ heterocycloalkyl, and C₃₋₁₂ heterocycloalkyl-C₁₋₆ alkyl. In some embodiments, R² is substituted with at least one of halogen, C₁₋₁₆ alkyl, C₁₋₁₆ haloalkyl, hydroxyl, C₁₋₁₆ alkoxy, C₁₋₁₆ haloalkoxy, amino, C₁₋₁₆alkylamino, or di-C₁₋₁₆alkylamino. In some embodiments, R⁴ is selected from the group consisting of 6-membered cycloalkyl, 6-membered heterocycloalkyl, 5-membered cycloalkyl, and 5-membered heterocycloalkyl. In some embodiments, R⁴ is selected from the group consisting of pyrimidine and pyridine. In some embodiments, R⁴ is substituted with at least one substituent selected from the group consisting of halogen, C₁₋₆ alkyl, C₁₋₆ haloalkyl, hydroxyl, C₁₋₆ alkoxy, C₁₋₆ haloalkoxy, amino, C₁₋₆ alkylamino, and di-C₁₋₆ alkylamino. In some embodiments, R⁴ is selected from the group consisting of: C₁₋₁₈ alkyl, alkenyl or alkynyl; C₀₋₁₈ alkyl, alkenyl or alkynyl-C₆₋₁₄ aryl; C₀₋₁₈ alkyl, alkenyl or alkynyl-C₃₋₁₂ cycloalkyl; C₀₋₁₈ alkyl, alkenyl or alkynyl-C₃₋₁₂ cycloalkyl-C₆₋₁₄ aryl; C₀₋₁₈ alkyl, alkenyl or alkynyl-di-C₆₋₁₄ aryl; C₀₋₁₈ alkyl, alkenyl or alkynyl-C₆₋₁₄ aryl-C₆₋₁₄ aryl; C₀₋₁₈ alkyl, alkenyl or alkynyl-C₆₋₁₄ aryl-O—C₆₋₁₄ aryl; and C₀₋₁₈ alkyl, alkenyl or alkynyl-C₆₋₁₄ aryl-C₂₋₅ N or S-heteroaryl. In some embodiments, R⁴ is selected from the group consisting of: C₆₋₁₄ n-alkyl; C₁₋₆ n-alkyl-phenyl, optionally substituted with a phenyl, C₁₋₃-alkyl-phenyl and —O-phenyl. In some embodiments, R³ is H. In some embodiments, R³ is selected from the group consisting of: C₁₋₁₈ alkyl, alkenyl or alkynyl; C₀₋₁₈ alkyl, alkenyl or alkynyl-C₆₋₁₄ aryl; C₀₋₁₈ alkyl, alkenyl or alkynyl-C₃₋₁₂ cycloalkyl; C₀₋₁₈ alkyl, alkenyl or alkynyl-C₃₋₁₂ cycloalkyl-C₆₋₁₄ aryl; C₀₋₁₈ alkyl, alkenyl or alkynyl-di-C₆₋₁₄ aryl; C₀₋₁₈ alkyl, alkenyl or alkynyl-C₆₋₁₄ aryl-C₆₋₁₄ aryl; C₀₋₁₈ alkyl, alkenyl or alkynyl-C₆₋₁₄ aryl-O—C₆₋₁₄ aryl; and C₀₋₁₈ alkyl, alkenyl or alkynyl-C₆₋₁₄ aryl-C₂₋₅ N, O or S-heteroaryl. In some embodiments, R¹ is H. In some embodiments, R² is selected from the group consisting of: C₁₋₁₈ alkyl, alkenyl or alkynyl; C₀₋₁₈ alkyl, alkenyl or alkynyl-C₆₋₁₄ aryl; C₀₋₁₈ alkyl, alkenyl or alkynyl-C₃₋₁₂ cycloalkyl; C₀₋₁₈ alkyl, alkenyl or alkynyl-C₃₋₁₂ cycloalkyl-C₆₋₁₄ aryl; C₀₋₁₈ alkyl, alkenyl or alkynyl-di-C₆₋₁₄ aryl; C₀₋₁₈ alkyl, alkenyl or alkynyl-C₆₋₁₄ aryl-C₆₋₁₄ aryl; C₀₋₁₈ alkyl, alkenyl or alkynyl-C₆₋₁₄ aryl-O—C₆₋₁₄ aryl; and C₀₋₁₈ alkyl, alkenyl or alkynyl-C₆₋₁₄ aryl-C₂₋₅ N, O or S-heteroaryl. In some embodiments, X and Z are CH₂.

Some embodiments of the disclosure include a compound shown in Table 1 below:

TABLE 1

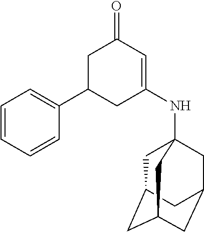 [SMILES: O=C1C=C(NC23CC4C[C@H](C2)C[C@@H](C4)C3)CC(c2ccccc2)C1]

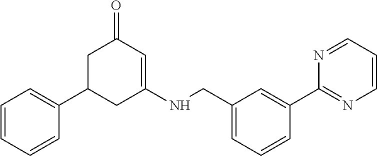 [SMILES: O=C1C=C(NCc2cccc(-c3ncccn3)c2)CC(c2ccccc2)C1]

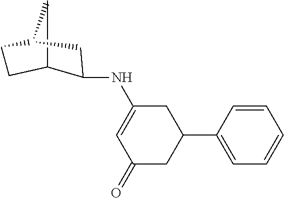

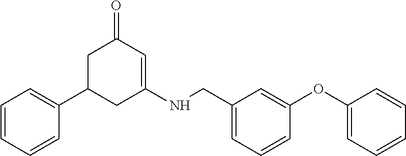 [SMILES: O=C1C=C(NCc2cccc(Oc3ccccc3)c2)CC(c2ccccc2)C1]

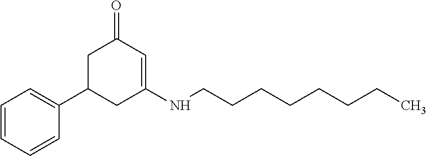 [SMILES: CCCCCCCCNC1=CC(=O)CC(c2ccccc2)C1]

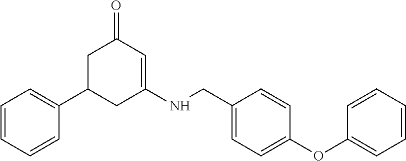 [SMILES: O=C1C=C(NCc2ccc(Oc3ccccc3)cc2)CC(c2ccccc2)C1]

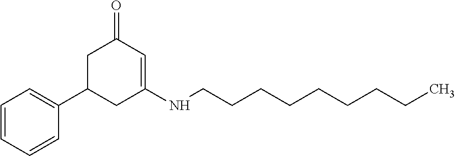 [SMILES: CCCCCCCCCNC1=CC(=O)CC(c2ccccc2)C1]

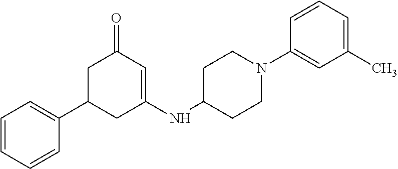 [SMILES: Cc1cccc(N2CCC(NC3=CC(=O)CC(c4ccccc4)C3)CC2)c1]

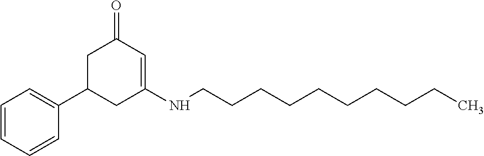 [SMILES: CCCCCCCCCCNC1=CC(=O)CC(c2ccccc2)C1]

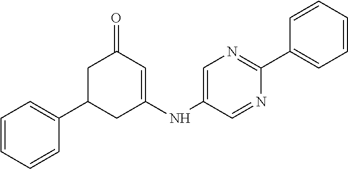 [SMILES: O=C1C=C(Nc2cnc(-c3ccccc3)nc2)CC(c2ccccc2)C1]

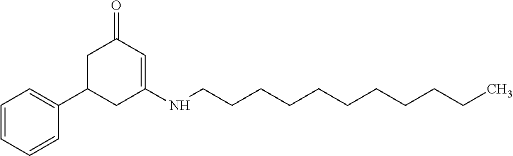 [SMILES: CCCCCCCCCCCNC1=CC(=O)CC(c2ccccc2)C1]

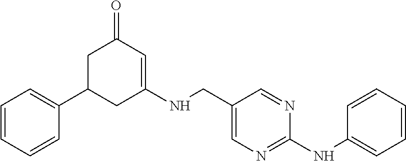 [SMILES: O=C1C=C(NCc2cnc(Nc3ccccc3)nc2)CC(c2ccccc2)C1]

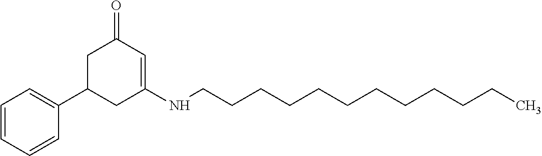 [SMILES: CCCCCCCCCCCCNC1=CC(=O)CC(c2ccccc2)C1]

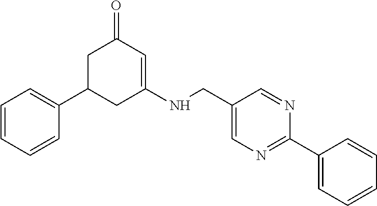 [SMILES: O=C1C=C(NCc2cnc(-c3ccccc3)nc2)CC(c2ccccc2)C1]

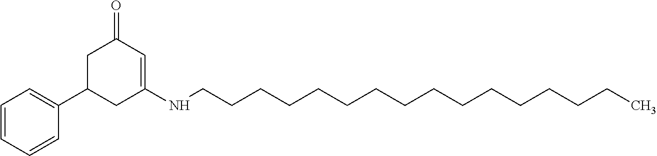 [SMILES: CCCCCCCCCCCCCCCCNC1=CC(=O)CC(c2ccccc2)C1]

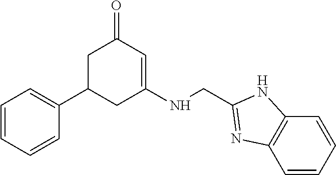 [SMILES: O=C1C=C(NCc2nc3ccccc3[nH]2)CC(c2ccccc2)C1]

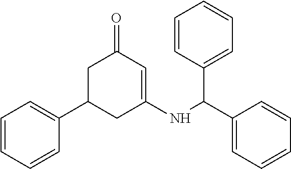 [SMILES: O=C1C=C(NC(c2ccccc2)c2ccccc2)CC(c2ccccc2)C1]

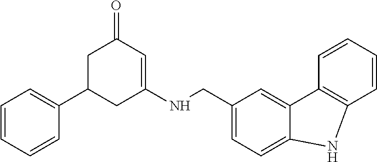 [SMILES: O=C1C=C(NCc2ccc3[nH]c4ccccc4c3c2)CC(c2ccccc2)C1]

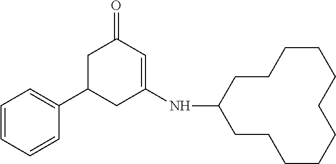 [SMILES: O=C1C=C(NC2CCCCCCCCCCC2)CC(c2ccccc2)C1]

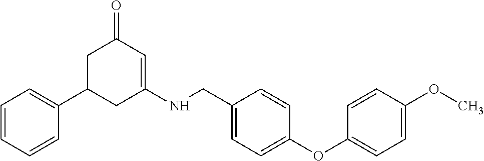 [SMILES: COc1ccc(Oc2ccc(CNC3=CC(=O)CC(c4ccccc4)C3)cc2)cc1]

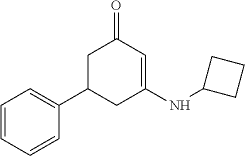 [SMILES: O=C1C=C(NC2CCC2)CC(c2ccccc2)C1]

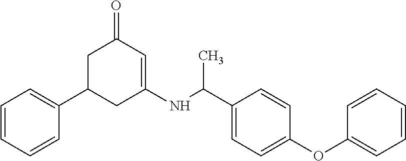 [SMILES: CC(NC1=CC(=O)CC(c2ccccc2)C1)c1ccc(Oc2ccccc2)cc1]

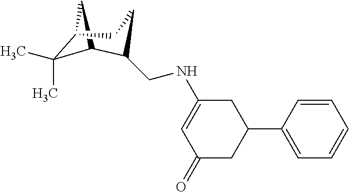

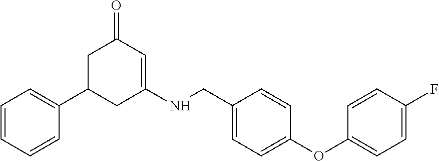 [SMILES: O=C1C=C(NCc2ccc(Oc3ccc(F)cc3)cc2)CC(c2ccccc2)C1]

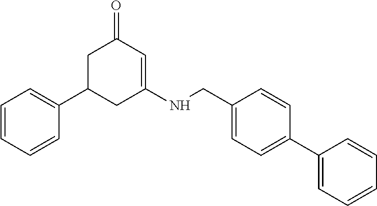 [SMILES: O=C1C=C(NCc2ccc(-c3ccccc3)cc2)CC(c2ccccc2)C1]

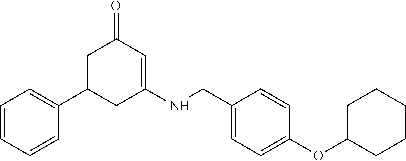 [SMILES: O=C1C=C(NCc2ccc(OC3CCCCC3)cc2)CC(c2ccccc2)C1]

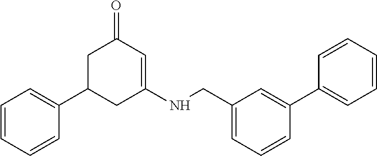 [SMILES: O=C1C=C(NCc2cccc(-c3ccccc3)c2)CC(c2ccccc2)C1]

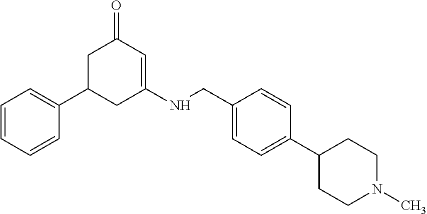 [SMILES: CN1CCC(c2ccc(CNC3=CC(=O)CC(c4ccccc4)C3)cc2)CC1]

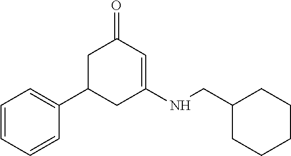 [SMILES: O=C1C=C(NCC2CCCCC2)CC(c2ccccc2)C1]

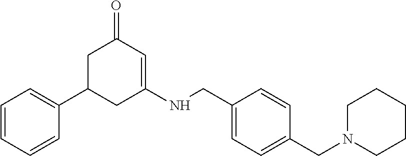 [SMILES: O=C1C=C(NCc2ccc(CN3CCCCC3)cc2)CC(c2ccccc2)C1]

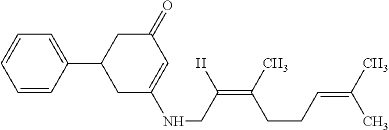 [SMILES: CC(C)=CCC/C(C)=C\CNC1=CC(=O)CC(c2ccccc2)C1]

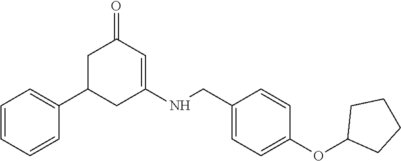 [SMILES: O=C1C=C(NCc2ccc(OC3CCCC3)cc2)CC(c2ccccc2)C1]

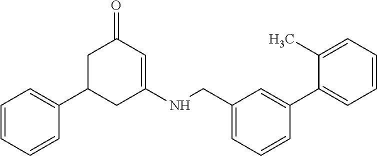 [SMILES: Cc1ccccc1-c1cccc(CNC2=CC(=O)CC(c3ccccc3)C2)c1]

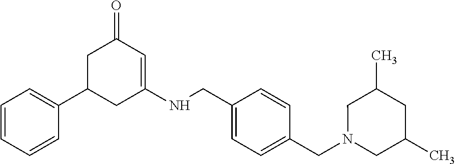 [SMILES: CC1CC(C)CN(Cc2ccc(CNC3=CC(=O)CC(c4ccccc4)C3)cc2)C1]

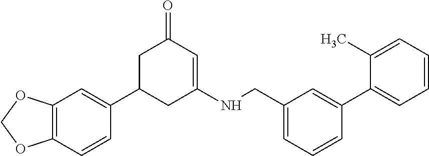 [SMILES: Cc1ccccc1-c1cccc(CNC2=CC(=O)CC(c3ccc4c(c3)OCO4)C2)c1]

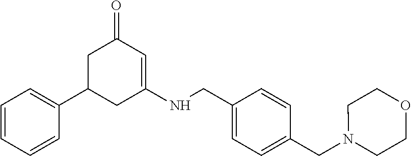 [SMILES: O=C1C=C(NCc2ccc(CN3CCOCC3)cc2)CC(c2ccccc2)C1]

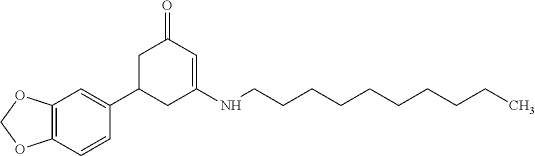 [SMILES: CCCCCCCCCCNC1=CC(=O)CC(c2ccc3c(c2)OCO3)C1]

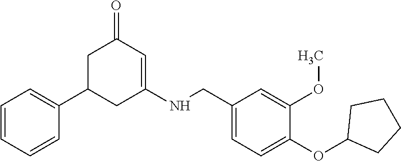 [SMILES: COc1cc(CNC2=CC(=O)CC(c3ccccc3)C2)ccc1OC1CCCC1]

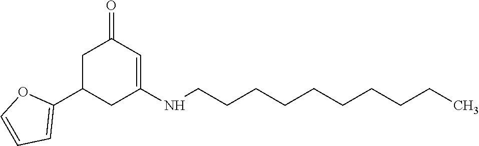 [SMILES: CCCCCCCCCCNC1=CC(=O)CC(c2ccco2)C1]

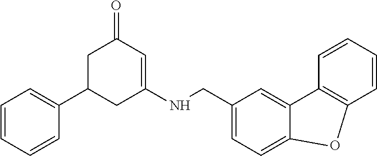 [SMILES: O=C1C=C(NCc2ccc3oc4ccccc4c3c2)CC(c2ccccc2)C1]

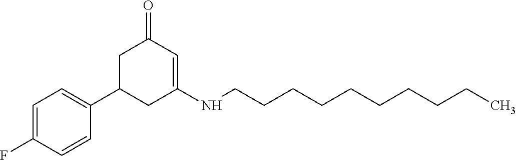 [SMILES: CCCCCCCCCCNC1=CC(=O)CC(c2ccc(F)cc2)C1]

Some embodiments of the disclosure include pharmaceutical compositions comprising a compound of the disclosure and a pharmaceutically acceptable excipient.

Other embodiments include a method of treating cancer in a patient in need thereof, the method comprising administering to the patient an effective amount of a compound according to formula (I):

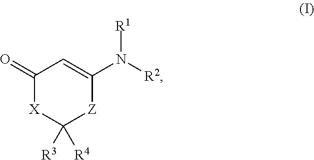

or a pharmaceutically acceptable salt, ester, hydrate or prodrug thereof, wherein:
X and Z are independently C₀₋₂-alkyl, —(CH₂)ₛ—NH—(CH₂)ₜ—, —(CH₂)ₛ—O—(CH₂)ₜ—, or —(CH₂)ₛ—C(NH₂)—(CH₂)ₜ—, wherein s and t are independently an integer of 0 or 1;
R¹ is selected from the group consisting of H, optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl;
R² is selected from the group consisting of optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl;
R³ is selected from the group consisting of H, optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl; and
R⁴ is selected from the group consisting of optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl,
wherein the cancer is selected from brain cancer, throat cancer, thyroid cancer, esophagus cancer, tonsil cancer, lung cancer, prostate cancer, colorectal cancer, stomach cancer, liver cancer, pancreatic cancer, gallbladder cancer, bladder cancer, rectal cancer, testicle cancer, breast cancer, cervical cancer, ovarian cancer, skin cancer, melanoma, leukemia, lymphoma, and multiple myeloma.

Other embodiments include a method of treating an acute and chronic inflammation disorder in a patient in need thereof, the method comprising administering to the patient an effective amount of a compound according to formula (I):

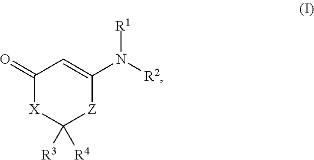

or a pharmaceutically acceptable salt, ester, hydrate or prodrug thereof, wherein:
X and Z are independently C₀₋₂-alkyl, —(CH₂)ₛ—NH—(CH₂)ₜ—, —(CH₂)ₛ—O—(CH₂)ₜ—, or —(CH₂)ₛ—C(NH₂)—(CH₂)ₜ—, wherein s and t are independently an integer of 0 or 1;
R¹ is selected from the group consisting of H, optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl;
R² is selected from the group consisting of optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl;
R³ is selected from the group consisting of H, optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl; and
R⁴ is selected from the group consisting of optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl.

In some embodiments, the acute and chronic inflammation disorder is selected from the group consisting of asthma, chronic obstructive lung disease, pulmonary fibrosis, pneumonitis (including hypersensitivity pneumonitis and radiation pneumonitis), pneumonia, cystic fibrosis, psoriasis, arthritis/rheumatoid arthritis, rhinitis, pharyngitis, cystitis, prostatitis, dermatitis, allergy including hay fever, nephritis, conjunctivitis, encephalitis, meningitis, opthalmitis, uveitis, pleuritis, pericarditis, myocarditis, atherosclerosis, human immunodeficiency virus related inflammation, diabetes, osteoarthritis, psoriatic arthritis, inflammatory bowel disease (Crohn's disease, ulcerative colitis)/colitis, sepsis, vasculitis, bursitis, connective tissue disease, autoimmune diseases such as systemic lupus erythematosis (SLE), polymyalgia rheumatica, scleroderma, Wegener's granulomatosis, temporal arteritis, vasculitis, cryoglobulinemia, and multiple sclerosis, viral or influenza-induced inflammation, and edema.

In some embodiments, the effective amount is from about 0.5 mg to about 500 mg of the compound of formula (I), or any numerical amount between these two values. In some embodiments, the effective amount is about 0.5 mg/kg to about 500 mg/kg of the compound of formula (I) per kg of patient body weight, or any numerical amount between these two values.

In some embodiments, the administration is oral administration, administration via implants, parenteral injection, intravenous injection, intraperitoneal injection, subcutaneous injection, bolus injection, infusion, rectal administration, vaginal administration, transdermal administration, inhalation, or any combination thereof. In some embodiments, the method further includes a step of administrating at least one anti-inflammatory agent, antimicrobial agent, matrix metalloprotease inhibitor, lipoxygenase inhibitor, cytokine antagonist, immunosuppressant, anti-cancer agent, anti-viral agent, cytokine, growth factor, immunomodulator, prostaglandin, anti-vascular hyperproliferation compound, and combinations thereof either concurrently with the compound of formula (I) or in the same course of treatment.

In some embodiments, the method is for treating sepsis, pneumonia, influenza-induced inflammation, edema, neuropathy, colitis, arthritis, Crohn's disease, diabetes, skin, eye and ear inflammation (e.g., psoriasis, uveitis/opthalmitis, external otitis), systemic lupus erythematosis (SLE), or systemic lupus erythematosis (SLE) in the patient in need thereof.

Other embodiments of the disclosure include use of a compound of formula (I) for treating acute and chronic inflammation disorders such as asthma, chronic obstructive lung disease, pulmonary fibrosis, pneumonitis (including hypersensitivity pneumonitis and radiation pneumonitis), pneumonia, cystic fibrosis, psoriasis, arthritis/rheumatoid arthritis, rhinitis, pharyngitis, cystitis, prostatitis, dermatitis, allergy including hay fever, nephritis, conjunctivitis, encephalitis, meningitis, opthalmitis, uveitis, pleuritis, pericarditis, myocarditis, atherosclerosis, human immunodeficiency virus related inflammation, diabetes, osteoarthritis, psoriatic arthritis, inflammatory bowel disease (Crohn's disease, ulcerative colitis)/colitis, sepsis, vasculitis, bursitis, connective tissue disease, autoimmune diseases such as systemic lupus erythematosis (SLE), polymyalgia rheumatica, scleroderma, Wegener's granulomatosis, temporal arteritis, vasculitis, cryoglobulinemia, and multiple sclerosis, viral or influenza-induced inflammation, or edema,

where formula (I) is:

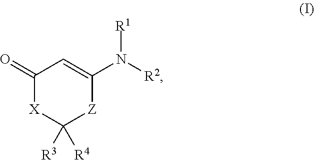

or a pharmaceutically acceptable salt, ester, hydrate thereof, wherein:
X and Z are independently C₀₋₂-alkyl, —(CH₂)ₛ—NH—(CH₂)ₜ—, —(CH₂)ₛ—O—(CH₂)ₜ—, or —(CH₂)ₛ—C(NH₂)—(CH₂)ₜ—, wherein s and t are independently an integer of 0 or 1;
R¹ is selected from the group consisting of H, optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl;
R² is selected from the group consisting of optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl;
R³ is selected from the group consisting of H, optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl; and
R⁴ is selected from the group consisting of optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl.

Other embodiments of the disclosure include use of a compound of formula (I) in the manufacture of a medicament for use in treating acute and chronic inflammation disorders such as asthma, chronic obstructive lung disease, pulmonary fibrosis, pneumonitis (including hypersensitivity pneumonitis and radiation pneumonitis), pneumonia, cystic fibrosis, psoriasis, arthritis/rheumatoid arthritis, rhinitis, pharyngitis, cystitis, prostatitis, dermatitis, allergy including hay fever, nephritis, conjunctivitis, encephalitis, meningitis, opthalmitis, uveitis, pleuritis, pericarditis, myocarditis, atherosclerosis, human immunodeficiency virus related inflammation, diabetes, osteoarthritis, psoriatic arthritis, inflammatory bowel disease (Crohn's disease, ulcerative colitis)/colitis, sepsis, vasculitis, bursitis, connective tissue disease, autoimmune diseases such as systemic lupus erythematosis (SLE), polymyalgia rheumatica, scleroderma, Wegener's granulomatosis, temporal arteritis, vasculitis, cryoglobulinemia, and multiple sclerosis, viral or influenza-induced inflammation, or edema,

wherein formula (I) is:

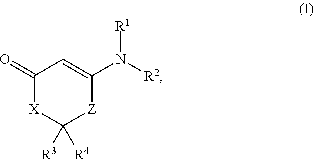

or a pharmaceutically acceptable salt, ester, hydrate thereof, wherein:
X and Z are independently C₀₋₂-alkyl, —(CH₂)ₛ—NH—(CH₂)ₜ—, —(CH₂)ₛ—O—(CH₂)ₜ—, or —(CH₂)ₛ—C(NH₂)—(CH₂)ₜ—, wherein s and t are independently an integer of 0 or 1;
R¹ is selected from the group consisting of H, optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl;
R² is selected from the group consisting of optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl;
R³ is selected from the group consisting of H, optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl; and
R⁴ is selected from the group consisting of optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl.

The foregoing general description and following brief description of the drawings and detailed description are exemplary and explanatory and are intended to provide further explanation of the invention as claimed. Other objects, advantages, and novel features will be readily apparent to those skilled in the art from the following brief description of the drawings and detailed description of the invention.

### BRIEF DESCRIPTION OF THE DRAWINGS

FIGS. 1A-1D show the following: FIGS. 1A-1B: DCN1 protein harbors a UBC12 binding pocket. In FIG. 1A, N-terminal of UBC12 is acetylated (yellow) and in FIG. 1B, it is completely buried inside the hydrophobic pocket within DCN1. FIG. 1C shows a candidate inhibitor 3-(pentylamino)-5-phenylcyclohex-2-en-1-one, and FIG. 1D shows a docking study of candidate inhibitor 3-(pentylamino)-5-phenylcyclohex-2-en-1-one with the DCN1 hydrophobic cavity.

FIG. 2A shows compounds used according to embodiments of the disclosure, and FIG. 2B shows an assay for protein immunoblotting after HepG2 cells were treated with compounds for 48 h at 10 uM, Cells were then collected and assayed for protein immunoblotting.

FIG. 3A shows compounds used according to embodiments of the disclosure, and FIG. 3B shows an assay for protein immunoblotting after HepG2 cells were treated with compounds for 48 h at various concentrations, cells were then collected and assayed for protein immunoblotting. FIG. 3C shows: Nedd8-cullin blots were quantified and graphed (n=3). FIG. 3D shows: 4K HepG2 cells were seeded in 96 wells plates, cells were then exposed to compounds at various concentrations for additional 96 h. Cell proliferation were then measured by CellTiter-Glo® Luminescent Cell Viability Assay (n=3).

FIG. 4A shows compounds used according to embodiments of the disclosure, and FIG. 4B shows an assay for protein immunoblotting after HepG2 cells were treated with 3-(decylamino)-5-phenylcyclohex-2-en-1-one, 5-(1,3-benzodioxol-5-yl)-3-(decylamino)cyclohex-2-en-1-one or MLN4924 for 48 h at various concentration. Cells were then collected and assayed for protein immunoblotting.

FIG. 5A shows shows an assay for protein immunoblotting after HepG2 cells were treated with 3-(decylamino)-5-phenylcyclohex-2-en-1-one for 48 h with various concentrations, cells were then collected and assayed for protein immunoblotting. FIG. 5B shows: Cell cycle analysis. HepG2 cells were treated with 3-(decylamino)-5-phenylcyclohex-2-en-1-one for 36 h with various concentrations. Cells were then analyzed by BrdU uptake and 7-AAD staining, G0, G1, S, G2/M phase cells were then quantitated and graphed. FIG. 5C shows: Wound-healing assay. Confluent monolayers of HepG2 cells were injured and cellular migration into the wound was determined under vehicle or 3-(decylamino)-5-phenylcyclohex-2-en-1-one treated condition. 36 h later cells were then observed under white field microscopy and recovery of cells to wound heal was quantified and graphed (right).

FIG. 6A shows the shows an assay for protein immunoblotting after HepG2 cells treated with 3-(decylamino)-5-phenylcyclohex-2-en-1-one at 10 μM for various times, and cells were collected and assayed for protein immunoblotting. FIG. 6B shows a cell cycle analysis. HepG2 cells were treated with 3-(decylamino)-5-phenylcyclohex-2-en-1-one at 10 μM for various times. At each time point, cells were analyzed by BrdU uptake and 7-AAD staining, G0, G1, S, G2/M phase cells were then quantitated and graphed. FIG. 6C shows: HepG2 cells treated with 3-(decylamino)-5-phenylcyclohex-2-en-1-one at 10 μM for various time. Cells were then observed under white field microscopy.

FIG. 7A shows a immunostained results and FIG. 7B shows immunostained results, each according to the following: HepG2 cells were seeded in 35 mm glass bottom dishes overnight and then treated with either vehicle or 3-(decylamino)-5-phenylcyclohex-2-en-1-one (1 uM) for additional 24 h. Cells were then washed with PBS and fixed with 4% paraformaldehyde for 20 min. Cells were co-immunostained for either α-tubulin. Cells were counterstained with DAPI to visualize the nucleus.

FIG. 8A shows mice tumor growth and FIG. 8B shows the effect of DCN1 inhibitor on growth of HepG2 tumor implants in nude mice. Mice were implanted with HepG2 cells (10⁶ cells) through subcutaneous injection. 2 weeks later, once tumor size reaches ˜0.1 cm³, mice were randomized and the treatment began by adding compounds to the drinking water (20 mg/kg/d). Tested compounds were: Vehicle, 3-(decylamino)-5-phenylcyclohex-2-en-1-one (BC1558), 3-(dibenzylamino)-5-phenylcyclohex-2-en-1-one (BC1634) or 3-((4-phenoxybenzyl)amino)-5-phenylcyclohex-2-en-1-one (BC1653). Tumor volume measurements over time were graphed (n=4-5 mice/group, *P<0.05 versus Vehicle).

FIG. 9A shows a timeline for the following experiment and FIG. 9B show the PD/PK model results of the experiment. Nude mice were implanted with HepG2 cells (10⁶ cells) through subcutaneous injection. Five weeks later, once tumor size reaches 0.3 cm³, mice were randomized and given 1 dose of 3-(decylamino)-5-phenylcyclohex-2-en-1-one (15 mg/kg) by oral gavage. Mice were then euthanized, plasma and tumor tissue were isolated for analysis. Tumor tissue were grinded and assayed for protein immunoblotting (n=3-4).

FIG. 10A shows the following: PBMC cells were cultured in 96 well plates before exposed to 3-(decylamino)-5-phenylcyclohex-2-en-1-one for 4 h. Cells were then treated with LPS (10 ng/ml) for additional 2 h. Media were then collected and TNF concentration was determined by ELISA. FIG. 10B shows: Fresh human lung slices were treated with 3-(decylamino)-5-phenylcyclohex-2-en-1-one for 18 h before exposed to LPS (100 ng/ml) for additional 4 h. Lung tissue was collected and assayed for immunoblotting. Culture media was collected and assayed for cytokines.

FIGS. 11A-11E show the Vehicle, 3-(decylamino)-5-phenylcyclohex-2-en-1-one, 3-(dibenzylamino)-5-phenylcyclohex-2-en-1-one and 3-((4-phenoxybenzyl)amino)-5-phenylcyclohex-2-en-1-one, which were each administered to mice though an i.p. injection (30 mg/kg), and mice were then immediately challenged with P. aeruginosa (strain PA103, 1.5*104 cfu/mouse, i.t.) for an additional 18 h. Mice were then euthanized and lungs were lavaged with saline, harvested, and then homogenized. FIG. 11A shows Lavage protein, FIG. 11B shows cell counts, FIG. 11C shows BAL IL 1b counts, FIG. 11D shows BAL IL6 counts, and FIG. 11E shows BAL TNF counts. The data represent n=4 mice/group, *P<0.05 versus Vehicle.

FIGS. 12A-12D show DCN1 inhibitors reduce DSS induced colonic inflammation, specifically the following experimental results C57BL6 mice were fed with water ad lib containing 3% dextran sulfate sodium (DSS) for six days. DCN1 inhibitors were administered into drinking water at 10 mg/kg/d, starting at day 1. Mice were euthanized at the end of the experiment: FIG. 12A: shows the length of the colon, FIG. 12B: shows the measured and graphed length of the colon. FIG. 12C: shows Colonic tissues cytokine IL1, and FIG. 12D: shows Colonic tissues cytokine IL6. The data represent n=4 mice/group, *P<0.05 versus CON.

### DETAILED DESCRIPTION

### I. Compounds of the Disclosure

Compounds of the present disclosure include novel compounds with the following core structure:

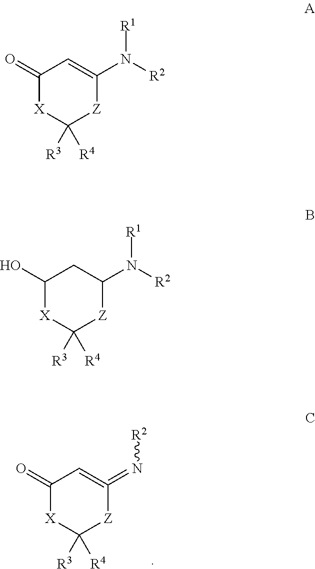

In some embodiments, the disclosure is related to novel compounds represented by formula (I), (II), or (III):

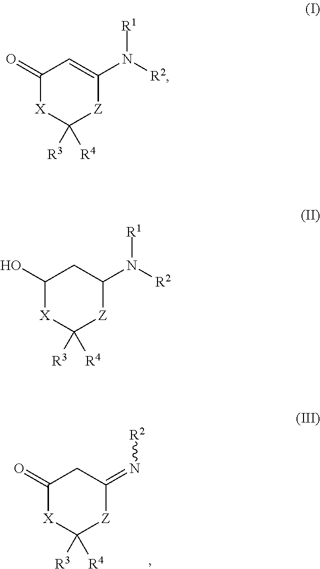

or a pharmaceutically acceptable salt, ester, hydrate or prodrug thereof, wherein:

X and Z are independently C₀₋₂-alkyl, —(CH₂)ₛ—NH—(CH₂)ₜ—, —(CH₂)ₛ—O—(CH₂)ₜ—, or —(CH₂)ₛ—C(NH₂)—(CH₂)ₜ—, wherein s and t are independently an integer of 0 or 1;

R¹ is selected from the group consisting of H, optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl;

R² is selected from the group consisting of optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl;

R³ is selected from the group consisting of H, optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl; and

R⁴ is selected from the group consisting of optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl.

In some embodiments, X and Z are each, individually, a bond, methyl, ethyl, —NH—, —O—, C(NH₂), CH₂—NH—, CH₂—O—, or —(CH₂—C(NH₂)—. In some embodiments, one or more of the hydrogens on the nitrogen is replaced by a protecting group.

In some embodiments, R¹ is H. In other embodiments, R¹ is one of the following: alkyl, alkenyl, alkynyl, alkoxy, aryl, cycloalkyl, heterocyclyl, alkoxy, aryl, cycloalkyl, heterocyclic, or heteroalkyl.

In some embodiments, R² is one of the following: alkyl, alkenyl, alkynyl, alkoxy, aryl, cycloalkyl, heterocyclyl, alkoxy, aryl, cycloalkyl, heterocyclic, or heteroalkyl. In some embodiments, R² is selected from the group consisting of C₃₋₁₂ cycloalkyl, C₃₋₁₂ cycloalkyl-C₁₋₆alkyl, C₃₋₁₂ heterocycloalkyl, or C₃₋₁₂ heterocycloalkyl-C₁₋₆ alkyl; or R² is substituted with at least one of halogen, C₁₋₁₆ alkyl, C₁₋₁₆haloalkyl, hydroxyl, C₁₋₁₆ alkoxy, C₁₋₁₆haloalkoxy, amino, C₁₋₁₆alkylamino, or di-C₁₋₁₆alkylamino; or R² is selected from the group consisting of C₁₋₁₆ alkyl, C₁₋₁₆ alkenyl, or C₁₋₁₆ alkynyl. In other embodiments, R² is selected from the group consisting of C₁₋₁₈ alkyl, alkenyl or alkynyl; C₀₋₁₈ alkyl, alkenyl or alkynyl-C₆₋₁₄ aryl; C₀₋₁₈ alkyl, alkenyl or alkynyl-C₃₋₁₂ cycloalkyl; C₀₋₁₈ alkyl, alkenyl or alkynyl-C₃₋₁₂ cycloalkyl-C₆₋₁₄ aryl; C₀₋₁₈ alkyl, alkenyl or alkynyl-di-C₆₋₁₄ aryl; C₀₋₁₈ alkyl, alkenyl or alkynyl-C₆₋₁₄ aryl-C₆₋₁₄ aryl; C₀₋₁₈ alkyl, alkenyl or alkynyl-C₆₋₁₄ aryl-O—C₆₋₁₄ aryl; or C₀₋₁₈ alkyl, alkenyl or alkynyl-C₆₋₁₄ aryl-C₂₋₅ N, O or S-heteroaryl. For example, some embodiments include where R² is optionally substituted C₀₋₁₈ alkyl-C₆₋₁₄ aryl (e.g., optionally substituted CH₂Ph), where the optional substitution may include, e.g., C₁₋₆ alkyl; C₁₋₆ perfluroalkyl; halogen; C₁₋₆ alkyl-aryl; C₁₋₆ alkyl-heteroaryl; C₁₋₆ alkyl-cycloalkyl; C₁₋₆ alkyl-heterocycle. Other optional substitution may include ethers, such as —O—C₁₋₆ alkyl; —O—C₁₋₆ perfluroalkyl; —O—C₁₋₆ alkyl-aryl; —O—C₁₋₆ alkyl-heteroaryl; —O—C₁₋₆ alkyl-cycloalkyl; —O—C₁₋₆ alkyl-heterocycle, each of which may be substituted by one or more C₁₋₆ alkyl; C₁₋₆ perfluroalkyl; halogen; —O—C₁₋₆ alkyl; —O—C₁₋₆ perfluroalkyl.

For example, in some embodiments, R² is one of the following:

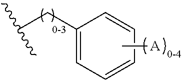

where A is halogen or A′-B′ where A′ is O or (CH₂)ₘ; m is 1-6, and B is H, C₁₋₆ alkyl; C₁₋₆ perfluroalkyl; halogen; aryl; heteroaryl; cycloalkyl; heterocycle, each of which may be substituted by 1, 2 or 3 halogen; C₁₋₆ alkyl; C₁₋₆ perfluroalkyl; or —O—C₁₋₆ alkyl. In some embodiments, the moiety contains one or more A (e.g., 1, 2 or 3 A's).

In some embodiments, R³ is H. In other embodiments, R³ is one of the following: alkyl, alkenyl, alkynyl, alkoxy, aryl, cycloalkyl, heterocyclyl, alkoxy, aryl, cycloalkyl, heterocyclic, or heteroalkyl. In some embodiments, R³ is an optionally substituted phenyl. The phenyl may be substituted by, e.g., halogen (F, Cl, Br, I), alkoxy (MeO, EtO, PrO, BuO, etc.), hydroxy. In some embodiments, the phenyl is substituted at adjacent positions to make a bicyclic moiety, for example, substituted by a fused phenyl, 5- or 6-membered cycloalkyl, 5- or 6-membered cycloalkyl substituted by 1 or 2 O, NH or S, or heterocyclyl. In some embodiments, R³ is selected from the group consisting of 6-membered cycloalkyl, 6-membered heterocycloalkyl, 5-membered cycloalkyl, and 5-membered heterocycloalkyl; or R³ is selected from the group consisting of pyrimidine and pyridine which may be optionally substituted, or R³ is substituted with at least one substituent selected from halogen, C₁₋₆ alkyl, C₁₋₆ haloalkyl, hydroxyl, C₁₋₆ alkoxy, C₁₋₆ haloalkoxy, amino, C₁₋₆ alkylamino, or di-C₁₋₆ alkylamino. In other embodiments, R³ is selected from the group consisting of: C₁₋₁₈ alkyl, alkenyl or alkynyl; C₀₋₁₈ alkyl, alkenyl or alkynyl-C₆₋₁₄ aryl; C₀₋₁₈ alkyl, alkenyl or alkynyl-C₃₋₁₂ cycloalkyl; C₀₋₁₈ alkyl, alkenyl or alkynyl-C₃₋₁₂ cycloalkyl-C₆₋₁₄ aryl; C₀₋₁₈ alkyl, alkenyl or alkynyl-di-C₆₋₁₄ aryl; C₀₋₁₈ alkyl, alkenyl or alkynyl-C₆₋₁₄ aryl-C₆₋₁₄ aryl; C₀₋₁₈ alkyl, alkenyl or alkynyl-C₆₋₁₄ aryl-O—C₆₋₁₄ aryl; C₀₋₁₈ alkyl, alkenyl or alkynyl-C₆₋₁₄ aryl-C₂₋₅ N or S-heteroaryl. In other embodiments, R³ is selected from the group consisting of: C₆₋₁₄ n-alkyl; C₁₋₆ n-alkyl-phenyl, optionally substituted with a phenyl, C₁₋₃-alkyl-phenyl or —O-phenyl.

In some embodiments, R⁴ is one of the following: alkyl, alkenyl, alkynyl, alkoxy, aryl, cycloalkyl, heterocyclyl, alkoxy, aryl, cycloalkyl, heterocyclic, or heteroalkyl. In some embodiments, R⁴ is an optionally substituted phenyl. The phenyl may be substituted by, e.g., halogen (F, Cl, Br, I), alkoxy (MeO, EtO, PrO, BuO, etc.), hydroxy. In some embodiments, the phenyl is substituted at adjacent positions to make a bicyclic moiety, for example, substituted by a fused phenyl, 5- or 6-membered cycloalkyl, 5- or 6-membered cycloalkyl substituted by 1 or 2 O, NH or S, or heterocyclyl. In some embodiments, R⁴ is selected from the group consisting of 6-membered cycloalkyl, 6-membered heterocycloalkyl, 5-membered cycloalkyl, and 5-membered heterocycloalkyl; or R⁴ is selected from the group consisting of pyrimidine and pyridine which may be optionally substituted, or R⁴ is substituted with at least one substituent selected from halogen, C₁₋₆ alkyl, C₁₋₆ haloalkyl, hydroxyl, C₁₋₆ alkoxy, C₁₋₆ haloalkoxy, amino, C₁₋₆ alkylamino, or di-C₁₋₆ alkylamino. In other embodiments, R⁴ is selected from the group consisting of: C₁₋₁₈ alkyl, alkenyl or alkynyl; C₀₋₁₈ alkyl, alkenyl or alkynyl-C₆₋₁₄ aryl; C₀₋₁₈ alkyl, alkenyl or alkynyl-C₃₋₁₂ cycloalkyl; C₀₋₁₈ alkyl, alkenyl or alkynyl-C₃₋₁₂ cycloalkyl-C₆₋₁₄ aryl; C₀₋₁₈ alkyl, alkenyl or alkynyl-di-C₆₋₁₄ aryl; C₀₋₁₈ alkyl, alkenyl or alkynyl-C₆₋₁₄ aryl-C₆₋₁₄ aryl; C₀₋₁₈ alkyl, alkenyl or alkynyl-C₆₋₁₄ aryl-O—C₆₋₁₄ aryl; C₀₋₁₈ alkyl, alkenyl or alkynyl-C₆₋₁₄ aryl-C₂₋₅ N or S-heteroaryl. In other embodiments, R⁴ is selected from the group consisting of: C₆₋₁₄ n-alkyl; C₁₋₆ n-alkyl-phenyl, optionally substituted with a phenyl, C₁₋₃-alkyl-phenyl or —O-phenyl.

In some embodiments, the disclosure is related to novel compounds represented by formula (I′), (I″), (IF), (II″), (III′), or (III″):

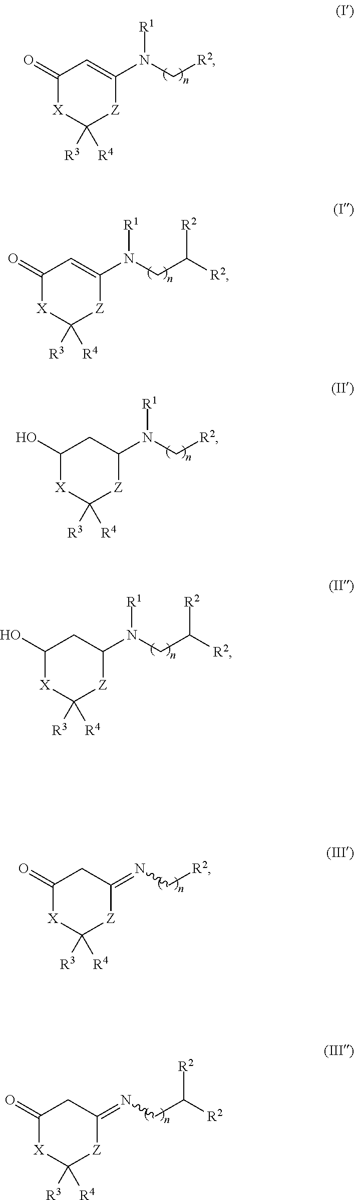

or a pharmaceutically acceptable salt, ester, hydrate or prodrug thereof, wherein n is 0, 1, 2, 3, 4, 5, 6, 7, 8, 9, or 10, and the remaining variables are the same as in Formulae (I), (II) and (III). In some embodiments, n is 0. In other embodiments, n is 1. In other embodiments, n is 2.

In some embodiments, the disclosure is related to novel compounds represented by formula (Ia), (Ib), (IIa), (IIb), (IIIa) or (IIIb):

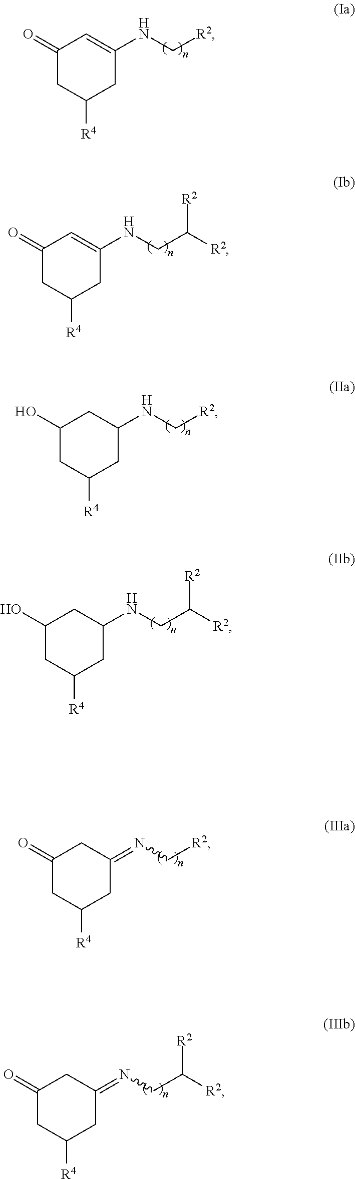

or a pharmaceutically acceptable salt, ester, hydrate or prodrug thereof, wherein the variables are the same as in formulae (I′) and (I″).

In some embodiments, the compounds of formulae (I), (I′) or (I″) do not include one or more of the following compounds shown in Table 2:

TABLE 2

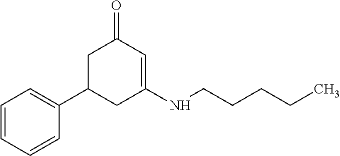 [SMILES: CCCCCNC1=CC(=O)CC(c2ccccc2)C1]

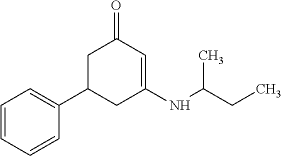 [SMILES: CCC(C)NC1=CC(=O)CC(c2ccccc2)C1]

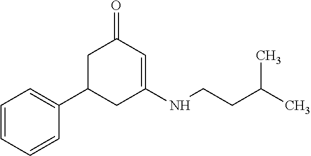 [SMILES: CC(C)CCNC1=CC(=O)CC(c2ccccc2)C1]

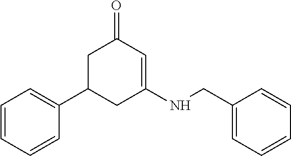 [SMILES: O=C1C=C(NCc2ccccc2)CC(c2ccccc2)C1]

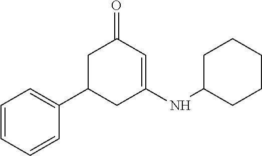 [SMILES: O=C1C=C(NC2CCCCC2)CC(c2ccccc2)C1]

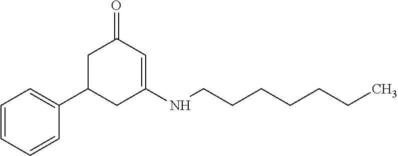 [SMILES: CCCCCCCNC1=CC(=O)CC(c2ccccc2)C1]

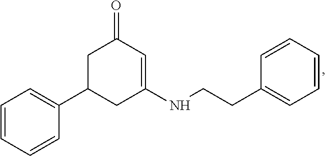 [SMILES: O=C1C=C(NCCc2ccccc2)CC(c2ccccc2)C1]

or a pharmaceutically acceptable salt, ester, hydrate or prodrug thereof. In some embodiments of formulae (I), (I′) or (I″), when X and Z are CH₂, and R¹ is H, R² is not an unsubstituted alkyl or an unsubstituted cycloalkyl or an unsubstituted aryl or an unsubstituted alkyl-aryl. For example, in some embodiments, when R¹ is H, R² is not an unsubstituted alkyl or an unsubstituted cycloalkyl or an unsubstituted aryl or an unsubstituted alkyl-aryl. In some embodiments of formulae (I), (I′) or (I″), when X and Z are CH₂ and R¹ is H, R² is not an unsubstituted C₁-C₈ alkyl (e.g., a C₃-C₇ alkyl) or an unsubstituted cyclohexyl or an unsubstituted phenyl or an unsubstituted C₁-C₂ alkyl-phenyl. For example, in some embodiments, when X and Z are CH₂ and R¹ is H, R² is not an unsubstituted alkyl or an unsubstituted cycloalkyl or an unsubstituted aryl or an unsubstituted alkyl-aryl.

In some embodiments, the compounds of the present disclosure are selected from the compounds of Table 3 (below) or a pharmaceutically acceptable salt thereof. It should be noted that the moieties of the compounds of Table 3 fall within the scope of compounds of Formulae (I), (II), (III), (I′), (I″), (IF), (II″), (III′), and (III″). The present disclosure includes embodiments where one or more of the variable moieties of Formulae (I), (II), (III), (I′), (I″), (IF), (II″), (III′), and (III″) are represented by the equivalent moiety of one or more of the compounds of Table 3 without requiring the other specific moieties of the same compound of Table 3.

### II. Methods of Treatment

In some embodiments, the compounds of the present disclosure are administered to a subject in need thereof as novel treatments to inhibit DCN1 or for novel treatments to suppress the growth of human tumor cells or for novel treatments to provide anti-inflammatory activity in vitro or in vivo or to provide anti-inflammatory activity in murine lung or colon inflammation. Specific treatments embodied herein include treating acute and chronic inflammation disorders such as asthma, chronic obstructive lung disease, pulmonary fibrosis, pneumonitis (including hypersensitivity pneumonitis and radiation pneumonitis), pneumonia, cystic fibrosis, psoriasis, arthritis/rheumatoid arthritis, rhinitis, pharyngitis, cystitis, prostatitis, dermatitis, allergy including hayfever, nephritis, conjunctivitis, encephalitis, meningitis, opthalmitis, uveitis, pleuritis, pericarditis, myocarditis, atherosclerosis, human immunodeficiency virus related inflammation, diabetes, osteoarthritis, psoriatic arthritis, inflammatory bowel disease (Crohn's disease, ulcerative colitis)/colitis, sepsis, vasculitis, bursitis, connective tissue disease, autoimmune diseases such as systemic lupus erythematosis (SLE), polymyalgia rheumatica, scleroderma, Wegener's granulomatosis, temporal arteritis, vasculitis, cryoglobulinemia, and multiple sclerosis, viral or influenza-induced inflammation, or edema, in a patent in need thereof, the method comprising administering an effective amount of a compound or composition of any of the disclosed embodiments. Additional treatments embodied herein include treating cancer in a patent in need thereof, the method comprising administering an effective amount of a compound or composition of any of the disclosed embodiments. In some embodiments, the cancer is selected from brain cancer, throat cancer, thyroid cancer, esophagus cancer, tonsil cancer, lung cancer, prostate cancer, colorectal cancer, stomach cancer, liver cancer, pancreatic cancer, gallbladder cancer, bladder cancer, rectal cancer, testicle cancer, breast cancer, cervical cancer, ovarian cancer, skin cancer, melanoma, leukemia, lymphoma, and multiple myeloma.

The compound may be included in a pharmaceutical formulation, such as those disclosed herein, and may be administered in any pharmaceutically acceptable manner, including methods of administration described herein. In some embodiments, the administration is an oral administration, administration via implants, parenteral injection, intravenous injection, intraperitoneal injection, subcutaneous injection, bolus injection, infusion, rectal administration, vaginal administration, transdermal administration, inhalation/pulmonary, nasal, and combinations thereof.

An effective amount of a compound useful in the methods of the present invention, for example in a pharmaceutical composition, may be administered to a mammal in need thereof by any of a number of well-known methods for administering pharmaceutical compounds. The compound may be administered systemically or locally. In one embodiment, the compound is administered intravenously. For example, the compounds useful in the methods of the present technology may be administered via rapid intravenous bolus injection. In some embodiments, the compound is administered as a constant rate intravenous infusion. The compound may also be administered orally, topically, intranasally, intramuscularly, subcutaneously, or transdermally. Other routes of administration include intracerebroventricularly or intrathecally. Intracerebroventiculatly refers to administration into the ventricular system of the brain. Intrathecally refers to administration into the space under the arachnoid membrane of the spinal cord.

In the above-embodied methods, the effective amount is from about 0.5 mg to about 500 mg of a compound disclosed herein, or any amount in between these two values, for example, about one of 0.5 mg, about 1, about 2, about 5, about 10, about 20, about 50, about 100, about 200, about 250, about 300, about 400, and about 500 mg. The effective amount may be an amount sufficient to alleviate or reduce one or more of the symptoms associated with the above-listed ailments. In some embodiments, the effective amount is about 0.5 mg/kg to about 500 mg/kg of compound per kg of patient body weight. In some embodiments, the effective amount is dosed per 8 hours, 12 hours, daily, or weekly.

The compounds useful in the methods of the present technology may also be administered to mammals by sustained or controlled release, as is known in the art. Sustained release administration is a method of drug delivery to achieve a certain level of the drug over a particular period of time. The level typically is measured by serum or plasma concentration.

In one preferred embodiment, the compounds are administered orally. In one preferred embodiment, the compounds are administered intravenously. In one preferred embodiment, the compounds are administered at less than 1 gram per day.

Some embodiments include a use of a compound or composition disclosed herein for treating acute and chronic inflammation disorders such as asthma, chronic obstructive lung disease, pulmonary fibrosis, pneumonitis (including hypersensitivity pneumonitis and radiation pneumonitis), pneumonia, cystic fibrosis, psoriasis, arthritis/rheumatoid arthritis, rhinitis, pharyngitis, cystitis, prostatitis, dermatitis, allergy including hayfever, nephritis, conjunctivitis, encephalitis, meningitis, opthalmitis, uveitis, pleuritis, pericarditis, myocarditis, atherosclerosis, human immunodeficiency virus related inflammation, diabetes, osteoarthritis, psoriatic arthritis, inflammatory bowel disease (Crohn's disease, ulcerative colitis)/colitis, sepsis, vasculitis, bursitis, connective tissue disease, autoimmune diseases such as systemic lupus erythematosis (SLE), polymyalgia rheumatica, scleroderma, Wegener's granulomatosis, temporal arteritis, vasculitis, cryoglobulinemia, and multiple sclerosis, viral or influenza-induced inflammation, or edema.

### III. Pharmaceutical Formulations

For oral administration, liquid or solid dose formulations may be used. Some examples of oral dosage formulations include tablets, gelatin capsules, pills, troches, elixirs, suspensions, syrups, wafers, chewing gum and the like. The compounds can be mixed with a suitable pharmaceutical carrier (vehicle) or excipient as understood by practitioners in the art. Examples of carriers and excipients include starch, milk, sugar, certain types of clay, gelatin, lactic acid, stearic acid or salts thereof, including magnesium or calcium stearate, talc, vegetable fats or oils, gums and glycols.

For systemic, intracerebroventricular, intrathecal, topical, intranasal, subcutaneous, or transdermal administration, formulations of the compounds useful in the methods of the present technology may utilize conventional diluents, carriers, or excipients etc., such as are known in the art can be employed to deliver the compounds. For example, the formulations may comprise one or more of the following: a stabilizer, a surfactant (such as a nonionic, ionic, anionic, or zwitterionic surfactant), and optionally a salt and/or a buffering agent. The compound may be delivered in the form of a solution or in a reconstituted lyophilized form.

In some embodiments, the stabilizer may, for example, be an amino acid, such as for instance, glycine; or an oligosaccharide, such as for example, sucrose, tetralose, lactose or a dextran. Alternatively, the stabilizer may be a sugar alcohol, such as for instance, mannitol; or a combination thereof. In some embodiments, the stabilizer or combination of stabilizers constitutes from about 0.1% to about 10% weight for weight of the compound.

In some embodiments, the surfactant is a nonionic surfactant, such as a polysorbate.

Some examples of suitable surfactants include polysorbates (e.g., Tween20, Tween80); a polyethylene glycol or a polyoxyethylene polyoxypropylene glycol, such as Pluronic F-68 at from about 0.001% (w/v) to about 10% (w/v).

A salt or buffering agent may be any salt or buffering agent, such as for example, sodium chloride, or sodium/potassium phosphate, respectively. In certain embodiments, the buffering agent maintains the pH of the pharmaceutical composition in the range of about 5.5 to about 7.5. The salt and/or buffering agent is also useful to maintain the osmolality at a level suitable for administration to a human or an animal. In some embodiments, the salt or buffering agent is present at a roughly isotonic concentration of about 150 mM to about 300 mM.

The formulations of the compounds useful in the methods of the present technology may additionally comprise one or more conventional additives. Some examples of such additives include a solubilizer such as, for example, glycerol; an antioxidant such as for example, benzalkonium chloride (a mixture of quaternary ammonium compounds, known as “quats”), benzyl alcohol, chloretone or chlorobutanol; anaesthetic agent such as for example a morphine derivative; or an isotonic agent etc., such as described above. As a further precaution against oxidation or other spoilage, the pharmaceutical compositions may be stored under nitrogen gas in vials sealed with impermeable stoppers.

The mammal can be any mammal, including, for example, farm animals, such as sheep, pigs, cows, and horses; pet animals, such as dogs and cats; laboratory animals, such as rats, mice and rabbits. In one embodiment, the mammal is a human.

Some embodiments include use of a compound disclosed herein in the manufacture of a medicament for use in treating acute and chronic inflammation disorders such as asthma, chronic obstructive lung disease, pulmonary fibrosis, pneumonitis (including hypersensitivity pneumonitis and radiation pneumonitis), pneumonia, cystic fibrosis, psoriasis, arthritis/rheumatoid arthritis, rhinitis, pharyngitis, cystitis, prostatitis, dermatitis, allergy including hayfever, nephritis, conjunctivitis, encephalitis, meningitis, opthalmitis, uveitis, pleuritis, pericarditis, myocarditis, atherosclerosis, human immunodeficiency virus related inflammation, diabetes, osteoarthritis, psoriatic arthritis, inflammatory bowel disease (Crohn's disease, ulcerative colitis)/colitis, sepsis, vasculitis, bursitis, connective tissue disease, autoimmune diseases such as systemic lupus erythematosis (SLE), polymyalgia rheumatica, scleroderma, Wegener's granulomatosis, temporal arteritis, vasculitis, cryoglobulinemia, and multiple sclerosis, viral or influenza-induced inflammation, or edema.

### IV. Combination Therapy

In some embodiments, the compounds of the present disclosure may be combined with one or more additional therapies that require one or more anti-inflammatory agents, antimicrobial agents, matrix metalloprotease inhibitors, lipoxygenase inhibitors, cytokine antagonists, immunosuppressants, anti-cancer agents, anti-viral agents, cytokines, growth factors, immunomodulators, prostaglandins, or an anti-vascular hyperproliferation compound.

The present disclosure includes combination therapies that include the method of any of the above disclosure, wherein the method further includes a step of administrating at least one anti-inflammatory agents, antimicrobial agents, matrix metalloprotease inhibitors, lipoxygenase inhibitors, cytokine antagonists, immunosuppressants, anti-cancer agents, anti-viral agents, cytokines, growth factors, immunomodulators, prostaglandins, anti-vascular hyperproliferation compounds, and combinations thereof either concurrently with the compound of Formula I or in the same course of treatment.

The multiple therapeutic agents may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents.

### V. Definitions

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely”, “only” and the like in connection with the recitation of claim elements, or use of a “negative” limitation.

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

Generally, reference to a certain element such as hydrogen or H is meant to include all isotopes of that element. For example, if an R group is defined to include hydrogen or H, it also includes deuterium and tritium. Hence, isotopically labeled compounds are within the scope of the invention.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group will be substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, alkynoxy, aryloxy, aralkyloxy, heterocyclyloxy, and heterocyclylalkoxy groups; carbonyls (oxo); carboxyls; esters; urethanes; oximes; hydroxylamines; alkoxy amines; aralkoxy amines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Substituted ring groups such as substituted cycloalkyl, aryl, heterocyclyl and heteroaryl groups also include rings and fused ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

“Alkyl” groups include straight chain and branched alkyl groups having from 1 to about 20 carbon atoms, and typically from 1 to 12 carbons or, in some embodiments, from 1 to 8, 1 to 6, or 1 to 4 carbon atoms. Alkyl groups further include cycloalkyl groups as defined below. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Cycloalkyl groups further include mono-, bicyclic and polycyclic ring systems, such as, for example bridged cycloalkyl groups as described below, and fused rings, such as, but not limited to, decalinyl, and the like. In some embodiments, polycyclic cycloalkyl groups have three rings. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Bridged cycloalkyl groups are cycloalkyl groups in which two or more hydrogen atoms are replaced by an alkylene bridge, wherein the bridge can contain 2 to 6 carbon atoms if two hydrogen atoms are located on the same carbon atom, or 1 to 5 carbon atoms, if the two hydrogen atoms are located on adjacent carbon atoms, or 2 to 4 carbon atoms if the two hydrogen atoms are located on carbon atoms separated by 1 or 2 carbon atoms. Bridged cycloalkyl groups can be bicyclic, such as, for example bicyclo[2.1.1]hexane, or tricyclic, such as, for example, adamantyl. Representative bridged cycloalkyl groups include bicyclo[2.1.1]hexyl, bicyclo[2.2.1]heptyl, bicyclo[3.2.1]octyl, bicyclo[2.2.2]octyl, bicyclo[3.2.2]nonyl, bicyclo[3.3.1]nonyl, bicyclo[3.3.2]decanyl, adamantyl, noradamantyl, bornyl, or norbornyl groups. Substituted bridged cycloalkyl groups may be substituted one or more times with non-hydrogen and non-carbon groups as defined above. Representative substituted bridged cycloalkyl groups may be mono-substituted or substituted more than once, such as, but not limited to, mono-, di- or tri-substituted adamantyl groups, which may be substituted with substituents such as those listed above.

Aryl groups are cyclic aromatic hydrocarbons that do not contain heteroatoms. Aryl groups include monocyclic, bicyclic and polycyclic ring systems. Thus, aryl groups include, but are not limited to, phenyl, azulenyl, heptalenyl, biphenylenyl, indacenyl, fluorenyl, phenanthrenyl, triphenylenyl, pyrenyl, naphthacenyl, chrysenyl, biphenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. In some embodiments, heterocyclyl groups include 3 to 20 ring members, whereas other such groups have 3 to 6, 3 to 10, 3 to 12, or 3 to 15 ring members. Heterocyclyl groups encompass unsaturated, partially saturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to as “substituted heterocyclyl groups”.

Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrohenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or di substituted with various substituents such as those listed above.

Heteroaryl groups are aromatic ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridyl, pyridazinyl, pyrimidinyl, pyrazinyl, thiophenyl, benzothiophenyl, furanyl, benzofuranyl, indolyl, azaindolyl (pyrrolopyridyl), indazolyl, benzimidazolyl, imidazopyridyl (azabenzimidazolyl), pyrazolopyridyl, triazolopyridyl, benzotriazolyl, benzoxazolyl, benzothiazolyl, benzothiadiazolyl, imidazopyridyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups. Although the phrase “heteroaryl groups” includes fused ring compounds such as indolyl and 2,3-dihydro indolyl, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

Groups described herein having two or more points of attachment (i.e., divalent, trivalent, or polyvalent) within the compound of the invention are designated by use of the suffix, “ene.” For example, divalent alkyl groups are alkylene groups, divalent aryl groups are arylene groups, divalent heteroaryl groups are divalent heteroaryl ene groups, and so forth. Substituted groups having a single point of attachment to the compound of the invention are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The term “amine” (or “amino”) as used herein refers to —NHR⁴ and —NR⁵R⁶ groups, wherein R⁴, R⁵ and R⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is NH₂, methylamino, dimethylamino, ethylamino, diethylamino, propylamino, isopropylamino, phenylamino, or benzylamino.

The term “amide” refers to a —NR′R″C(O)— group wherein R′ and R″ each independently refer to a hydrogen, (C₁-C₈)alkyl, or (C₃-C₆)aryl.

The term ‘nitrile or cyano” can be used interchangeably and refer to a —CN group which is bound to a carbon atom of a heteroaryl ring, aryl ring and a heterocycloalkyl ring.

The substituent —CO₂H, may be replaced with bioisosteric replacements such as:

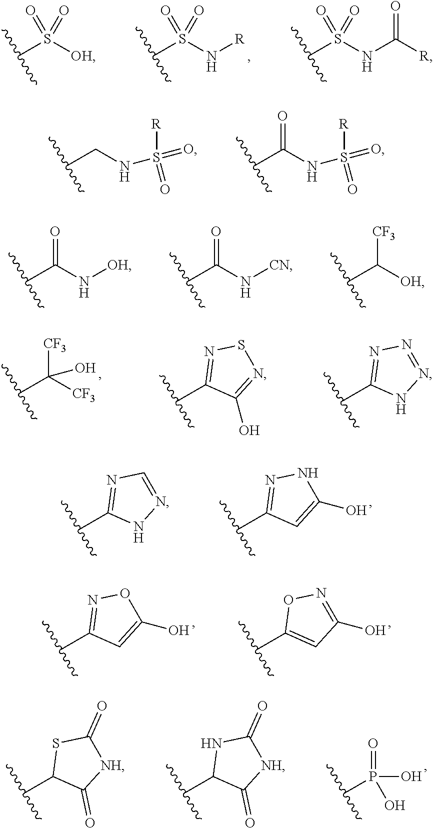

and the like, wherein R has the same definition as R′ and R″ as defined herein. See, e.g., THE PRACTICE OF MEDICINAL CHEMISTRY (Academic Press: New York, 1996), at page 203.

Those of skill in the art will appreciate that compounds of the invention may exhibit the phenomena of tautomerism, conformational isomerism, geometric isomerism and/or optical isomerism. As the formula drawings within the specification and claims can represent only one of the possible tautomeric, conformational isomeric, optical isomeric or geometric isomeric forms, it should be understood that the invention encompasses any tautomeric, conformational isomeric, optical isomeric and/or geometric isomeric forms of the compounds having one or more of the utilities described herein, as well as mixtures of these various different forms.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

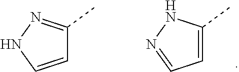

As readily understood by one skilled in the art, a wide variety of functional groups and other structures may exhibit tautomerism, and all tautomers of compounds as described herein are within the scope of the present invention.

Stereoisomers of compounds, also known as “optical isomers,” include all chiral, diastereomeric, and racemic forms of a structure, unless the specific stereochemistry is expressly indicated. Thus, compounds used in the present invention include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these are all within the scope of the invention.

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U S. and Drug administration.

By “patient” is meant any animal for which treatment is desirable. Patients may be mammals, and typically, as used herein, a patient is a human individual.

The term “pharmaceutically acceptable salt,” as used herein, represents salts or zwitterionic forms of the compounds of the present invention which are water or oil-soluble or dispersible; which are suitable for treatment of diseases without undue toxicity, irritation, and allergic-response; which are commensurate with a reasonable benefit/risk ratio; and which are effective for their intended use. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), hi sulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, pi crate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form pharmaceutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds of the compounds of the present invention and the like.

The term “solvates” is used in its broadest sense. For example, the term solvates includes hydrates formed when a compound of the present invention contains one or more bound water molecules.

Certain ranges are presented herein with numerical values being preceded by the term “about”. The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

This disclosure is not limited to particular embodiments described, as such may, of course, vary. It is also to be understood that the terminology used herein is for the purpose of describing particular embodiments only, and is not intended to be limiting, since the scope of the present invention will be limited only by the appended claims.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

### VI. Working Examples

The present technology is further illustrated by the following examples, which should not be construed as limiting in any way.

General Information.

All non-aqueous reactions were carried out under a nitrogen atmosphere in oven- or flame-dried glassware unless otherwise noted. Anhydrous tetrahydrofuran and diethyl ether were distilled from sodium benzophenone ketyl; anhydrous dichloromethane and toluene were distilled from CaH₂; alternatively, the same solvents were obtained from a solvent purification system using alumina columns. All other solvents and reagents were used as obtained from commercial sources without further purification unless noted. Reactions were monitored via TLC using 250 μm pre-coated silica gel 60 F₂₅₄ plates, which were visualized with 254 nm and/or 365 nm UV light and by staining with KMnO₄ (1.5 g KMnO₄, 10 g K₂CO₃, and 1.25 mL 10% NaOH in 200 mL water), cerium molybdate (0.5 g Ce(NH₄)₂(NO₃)₆, 12 g (NH₄)₆Mo₇O₂₄.4H₂O, and 28 mL cone. H₂SO₄ in 235 mL water), or vanillin (6 g vanillin and 1.5 mL cone. H₂SO₄ in 100 mL EtOH). Flash chromatography was performed with SiliCycle silica gel 60 (230-400 mesh) or with ISCO MPLC. ¹H and ¹³C NMR spectra were recorded on Bruker Avance 300, 400, or 500 MHz spectrometers, using the residual solvent as an internal standard. IR spectra were obtained on a Smiths IdentifyIR or PerkinElmer Spectrum 100. HRMS data were obtained on a Thermo Scientific Exactive HRMS coupled to a Thermo Scientific Accela HPLC system using a 2.1×50 mm 3.5 μm Waters XTerra C₁₈ column eluting with MeCN/H₂O containing 0.1% formic acid. Purity of compounds was assessed using the same HPLC system with either the PDA or an Agilent 385 ELSD. All final screening samples passed QC based on >95% purity by LC/MS/ELSD analysis.

General Synthetic Scheme

Generally, equimolar amounts (e.g., 5 mmol) of amine variant and cyclohexane-1,3-dione variant were added to a suitable solvent (e.g., 30 ml ethyl acetate). The solution was stirred and refluxed for, e.g., 30 min. Progression of the reaction was monitored by techniques known in the art. The reaction mixture was cooled in an ice bath, and the resulting product was precipitated, filtered, and dried to provide the desired products. Products can be further purified by crystallization or column chromatography, as is customary in the art.

### Example 1

5 mmol of Decylamine and 5 mmol of 5-Phenylcyclohexane-1,3-di one were added to 30 ml ethyl acetate. The solution was stirred and refluxed for 30 min. The reaction mixture was cooled in an ice bath, and the resulting product was precipitated, filtered, dried to desired products as yellow powder (0.95 g, 55% yield).

Additional compounds were synthesized, and are shown in Table 3.

### Example 2

4KHepG2 cells were seeded in 96 well plates for overnight. Compounds listed in Table 3 were then added to each well at various concentrations. 96 h later, cell proliferation was measured by ATP production using a CellTiter-Glo® Luminescent Cell Viability Assay (Promega). IC50_{Proliferation} for each compound was determined (n=3), and is shown in Table 3.

TABLE 3

| | Compound | IC50_{Proliferation} |
|---|---|---|
| BC1526 | 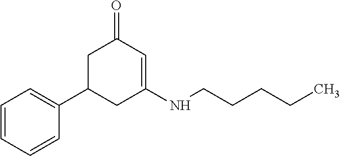 [SMILES: CCCCCNC1=CC(=O)CC(c2ccccc2)C1] | ++ |
| BC1532 | 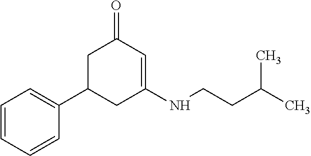 [SMILES: CC(C)CCNC1=CC(=O)CC(c2ccccc2)C1] | ++ |
| BC1534 | 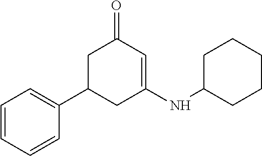 [SMILES: O=C1C=C(NC2CCCCC2)CC(c2ccccc2)C1] | + |
| BC1535 | 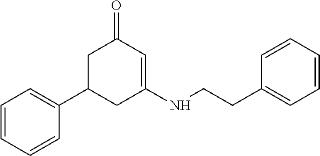 [SMILES: O=C1C=C(NCCc2ccccc2)CC(c2ccccc2)C1] | ++ |
| BC1546 | 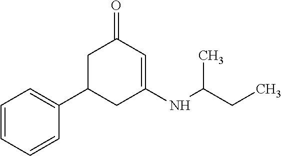 [SMILES: CCC(C)NC1=CC(=O)CC(c2ccccc2)C1] | + |
| BC1548 | 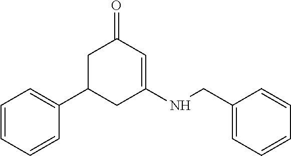 [SMILES: O=C1C=C(NCc2ccccc2)CC(c2ccccc2)C1] | ++ |
| BC1549 | 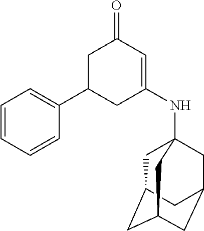 [SMILES: O=C1C=C(NC23CC4C[C@H](C2)C[C@@H](C4)C3)CC(c2ccccc2)C1] | + |
| BC1550 | 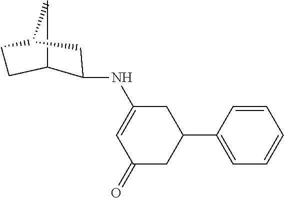 | ++ |
| BC1552 | 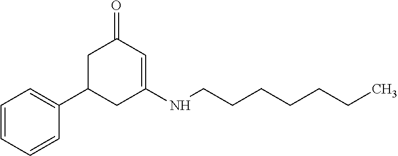 [SMILES: CCCCCCCNC1=CC(=O)CC(c2ccccc2)C1] | +++ |
| BC1554 | 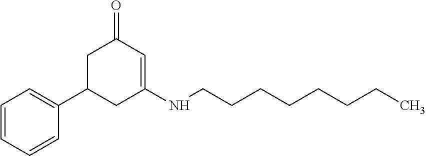 [SMILES: CCCCCCCCNC1=CC(=O)CC(c2ccccc2)C1] | +++ |
| BC1556 | 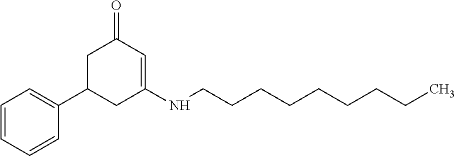 [SMILES: CCCCCCCCCNC1=CC(=O)CC(c2ccccc2)C1] | +++ |
| BC1558 | 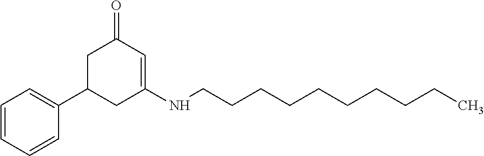 [SMILES: CCCCCCCCCCNC1=CC(=O)CC(c2ccccc2)C1] | +++ |
| BC1560 | 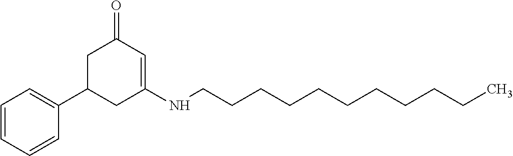 [SMILES: CCCCCCCCCCCNC1=CC(=O)CC(c2ccccc2)C1] | +++ |
| BC1562 | 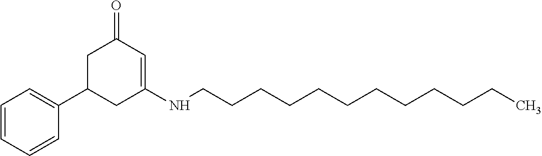 [SMILES: CCCCCCCCCCCCNC1=CC(=O)CC(c2ccccc2)C1] | +++ |
| BC1564 | 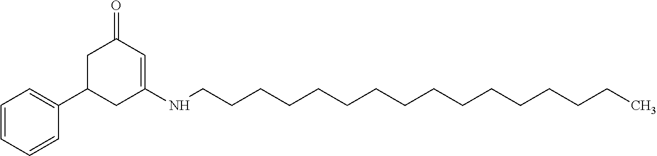 [SMILES: CCCCCCCCCCCCCCCCNC1=CC(=O)CC(c2ccccc2)C1] | + |
| BC1621 | 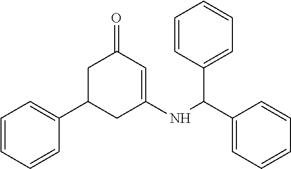 [SMILES: O=C1C=C(NC(c2ccccc2)c2ccccc2)CC(c2ccccc2)C1] | + |
| BC1623 | 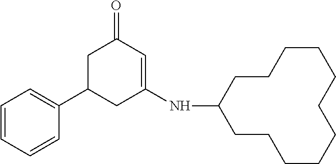 [SMILES: O=C1C=C(NC2CCCCCCCCCCC2)CC(c2ccccc2)C1] | + |
| BC1625 | 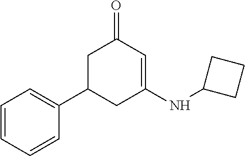 [SMILES: O=C1C=C(NC2CCC2)CC(c2ccccc2)C1] | + |
| BC1627 | 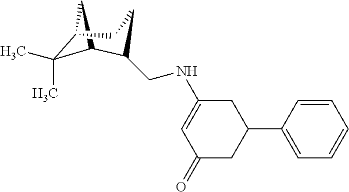 | ++ |
| BC1629 | 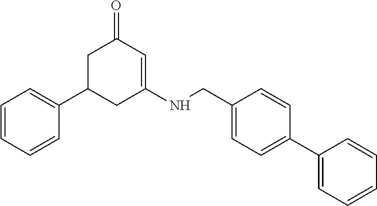 [SMILES: O=C1C=C(NCc2ccc(-c3ccccc3)cc2)CC(c2ccccc2)C1] | +++ |
| BC1631 | 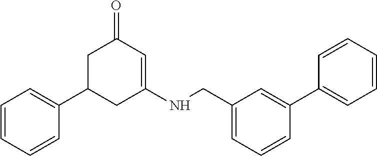 [SMILES: O=C1C=C(NCc2cccc(-c3ccccc3)c2)CC(c2ccccc2)C1] | +++ |
| BC1633 | 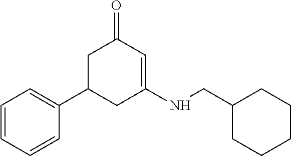 [SMILES: O=C1C=C(NCC2CCCCC2)CC(c2ccccc2)C1] | + |
| BC1634 | 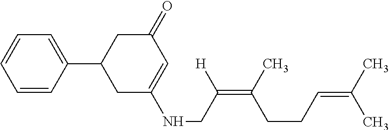 [SMILES: CC(C)=CCC/C(C)=C\CNC1=CC(=O)CC(c2ccccc2)C1] | +++ |
| BC1640 | 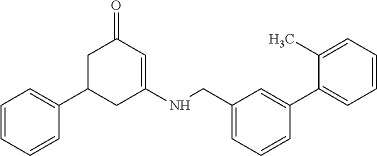 [SMILES: Cc1ccccc1-c1cccc(CNC2=CC(=O)CC(c3ccccc3)C2)c1] | ++ |
| BC1641 | 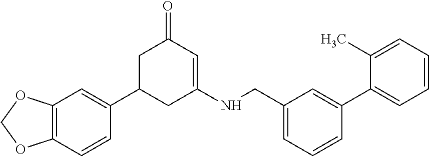 [SMILES: Cc1ccccc1-c1cccc(CNC2=CC(=O)CC(c3ccc4c(c3)OCO4)C2)c1] | + |
| BC1644 | 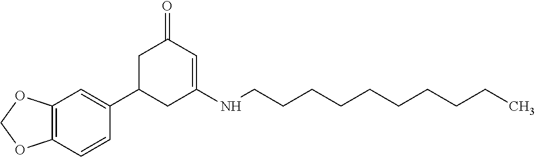 [SMILES: CCCCCCCCCCNC1=CC(=O)CC(c2ccc3c(c2)OCO3)C1] | + |
| BC1646 | 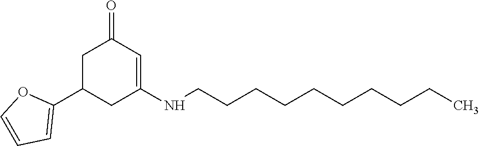 [SMILES: CCCCCCCCCCNC1=CC(=O)CC(c2ccco2)C1] | +++ |
| BC1648 | 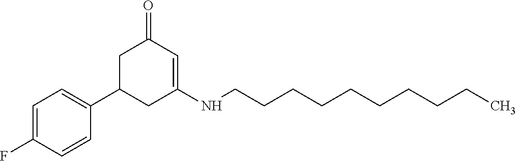 [SMILES: CCCCCCCCCCNC1=CC(=O)CC(c2ccc(F)cc2)C1] | ++ |
| BC1650 | 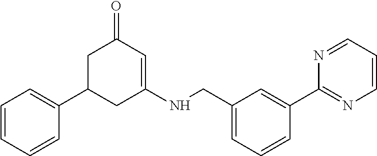 [SMILES: O=C1C=C(NCc2cccc(-c3ncccn3)c2)CC(c2ccccc2)C1] | ++ |
| BC1651 | 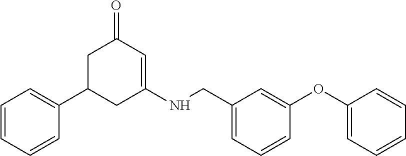 [SMILES: O=C1C=C(NCc2cccc(Oc3ccccc3)c2)CC(c2ccccc2)C1] | +++ |
| BC1653 | 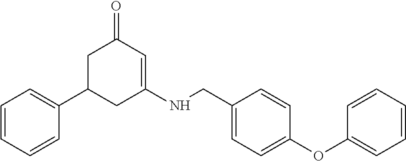 [SMILES: O=C1C=C(NCc2ccc(Oc3ccccc3)cc2)CC(c2ccccc2)C1] | +++ |
| BC1654 | 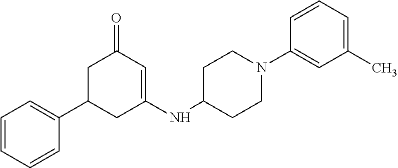 [SMILES: Cc1cccc(N2CCC(NC3=CC(=O)CC(c4ccccc4)C3)CC2)c1] | + |
| BC1655 | 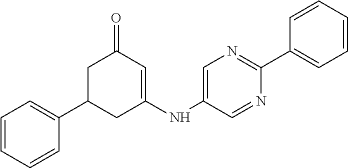 [SMILES: O=C1C=C(Nc2cnc(-c3ccccc3)nc2)CC(c2ccccc2)C1] | + |
| BC1656 | 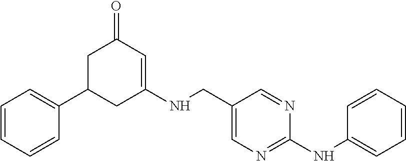 [SMILES: O=C1C=C(NCc2cnc(Nc3ccccc3)nc2)CC(c2ccccc2)C1] | + |
| BC1657 | 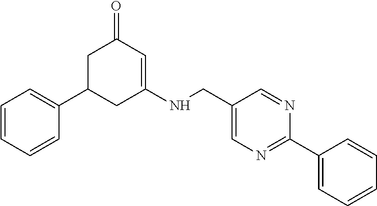 [SMILES: O=C1C=C(NCc2cnc(-c3ccccc3)nc2)CC(c2ccccc2)C1] | ++ |
| BC1658 | 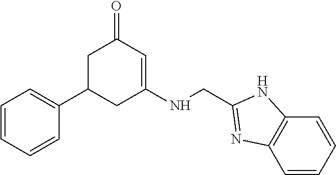 [SMILES: O=C1C=C(NCc2nc3ccccc3[nH]2)CC(c2ccccc2)C1] | + |
| BC1659 | 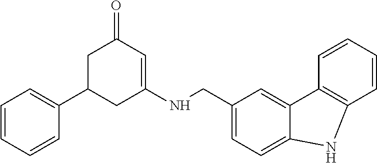 [SMILES: O=C1C=C(NCc2ccc3[nH]c4ccccc4c3c2)CC(c2ccccc2)C1] | +++ |
| BC1660 | 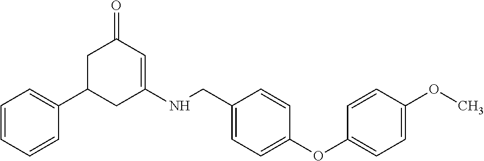 [SMILES: COc1ccc(Oc2ccc(CNC3=CC(=O)CC(c4ccccc4)C3)cc2)cc1] | ++ |
| BC1661 | 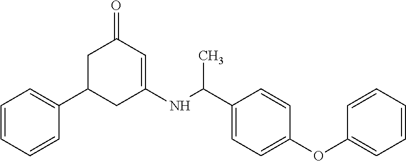 [SMILES: CC(NC1=CC(=O)CC(c2ccccc2)C1)c1ccc(Oc2ccccc2)cc1] | + |
| BC1662 | 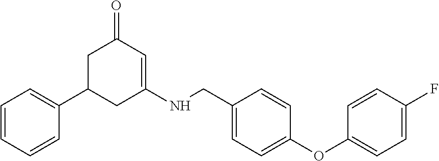 [SMILES: O=C1C=C(NCc2ccc(Oc3ccc(F)cc3)cc2)CC(c2ccccc2)C1] | +++ |
| BC1663 | 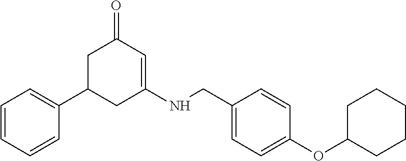 [SMILES: O=C1C=C(NCc2ccc(OC3CCCCC3)cc2)CC(c2ccccc2)C1] | +++ |
| BC1664 | 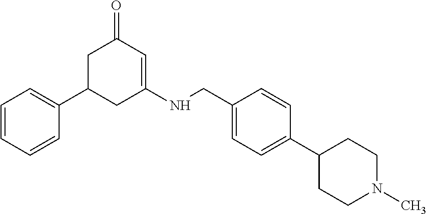 [SMILES: CN1CCC(c2ccc(CNC3=CC(=O)CC(c4ccccc4)C3)cc2)CC1] | + |
| BC1665 | 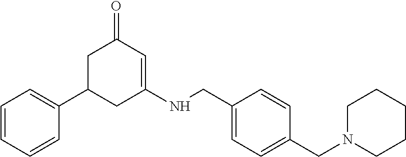 [SMILES: O=C1C=C(NCc2ccc(CN3CCCCC3)cc2)CC(c2ccccc2)C1] | + |
| BC1666 | 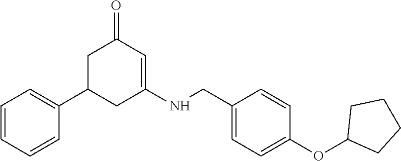 [SMILES: O=C1C=C(NCc2ccc(OC3CCCC3)cc2)CC(c2ccccc2)C1] | +++ |
| BC1667 | 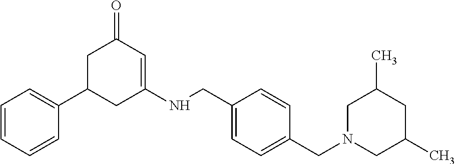 [SMILES: CC1CC(C)CN(Cc2ccc(CNC3=CC(=O)CC(c4ccccc4)C3)cc2)C1] | + |
| BC1668 | 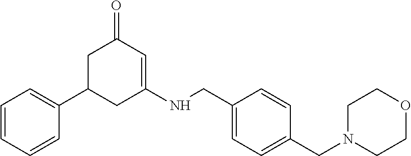 [SMILES: O=C1C=C(NCc2ccc(CN3CCOCC3)cc2)CC(c2ccccc2)C1] | ++ |
| BC1669 | 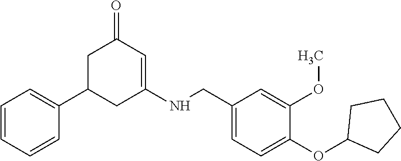 [SMILES: COc1cc(CNC2=CC(=O)CC(c3ccccc3)C2)ccc1OC1CCCC1] | +++ |
| BC1670 | 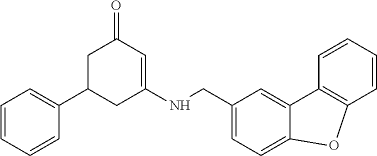 [SMILES: O=C1C=C(NCc2ccc3oc4ccccc4c3c2)CC(c2ccccc2)C1] | ++ |

*“+” > 10μM, “++” > 1μM and < 10μM, “+++” < 1μM.

### Example 3

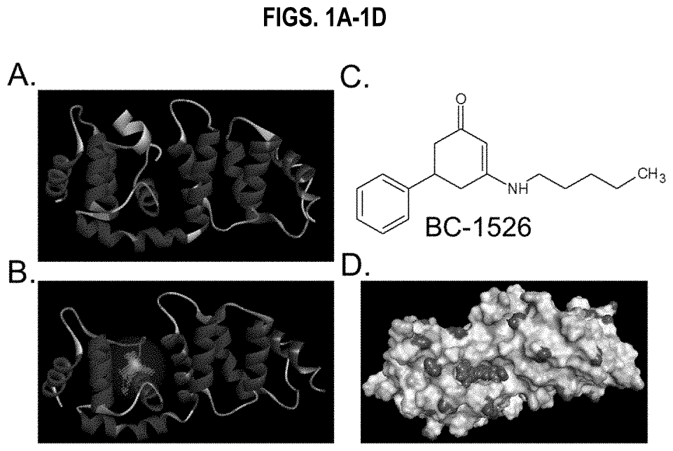

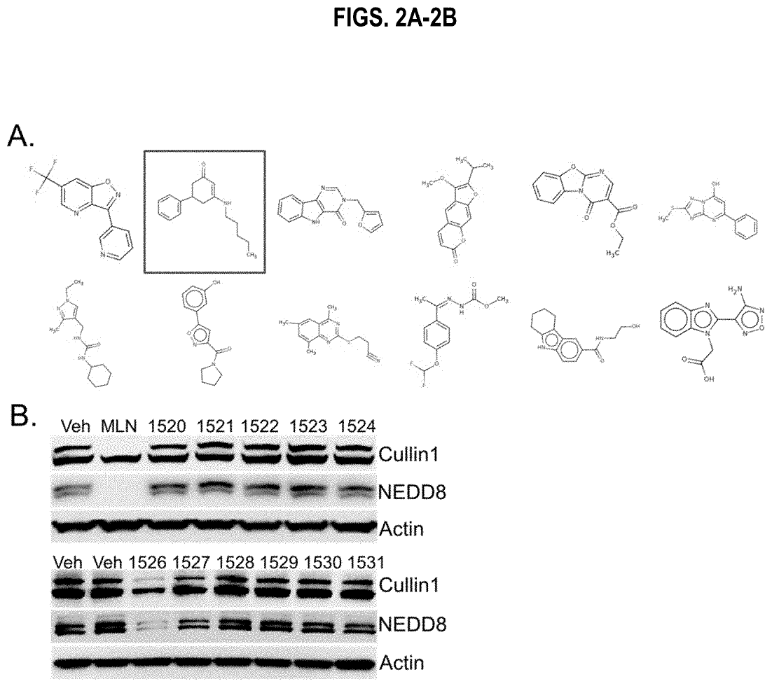

DCN1 protein is believed to harbor a UBC12 binding pocket. Scott et al, Cell 757:1671-84 (2014). The structure of a RING E3 trapped in action reveals that ligation mechanism for the ubiquitin-like protein NEDD8. Scott et al. (2014). The N-terminal acetylation acts as an avidity enhancer within an interconnected multiprotein complex. Science, 554:674-8 (2011). Specifically, n-terminal of UBC12 is acetylated (FIG. 1A, yellow) and completely buried inside the hydrophobic pocket within DCN1 (4gao.PDB, FIG. 1B). Small molecule inhibition of the DCN1 hydrophobic pocket would presumably disrupt UBC12 binding, and thus disrupt Neddylation of Cullin protein. Potential ligands were assessed that might fit the DCN1 domain cavities (FIG. 1B). 3-(pentylamino)-5-phenylcyclohex-2-en-1-one was selected for future testing (FIG. 1C). FIG. 1D shows the in silico docking model 3-(pentylamino)-5-phenylcyclohex-2-en-1-one (Green) bound to DCN1. 3-(pentylamino)-5-phenylcyclohex-2-en-1-one showed good activity towards reducing cullin1 neddylation (FIG. 2). MLN4924 was used as a positive control.

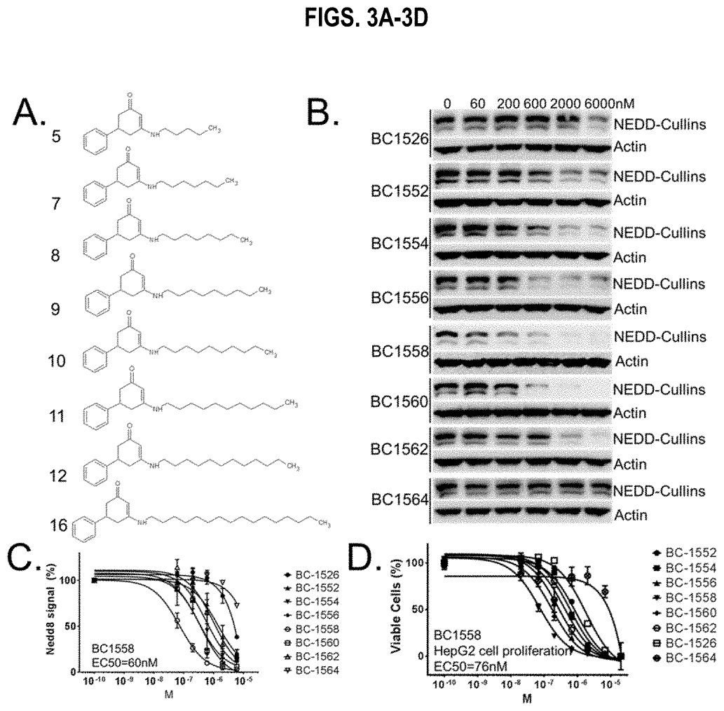

3-(pentylamino)-5-phenylcyclohex-2-en-1-one was modified by adding a different sidechain. These novel compounds were tested further in an in vitro proliferation assay using ATPLite Cell Proliferation kit. The compound structures and their IC50_{proliferation} are listed in Table 3. Certain compounds showed significant improvement in their activity (FIG. 3A-B). Others lost activity in reducing Cullin Neddylation and inhibiting cell proliferation (FIG. 3C-D). 3-(decylamino)-5-phenylcyclohex-2-en-1-one which harbors CIO alkyl sidechain exhibited activity in reducing Cullin Neddylation and inhibiting cell proliferation (FIG. 3C-D).

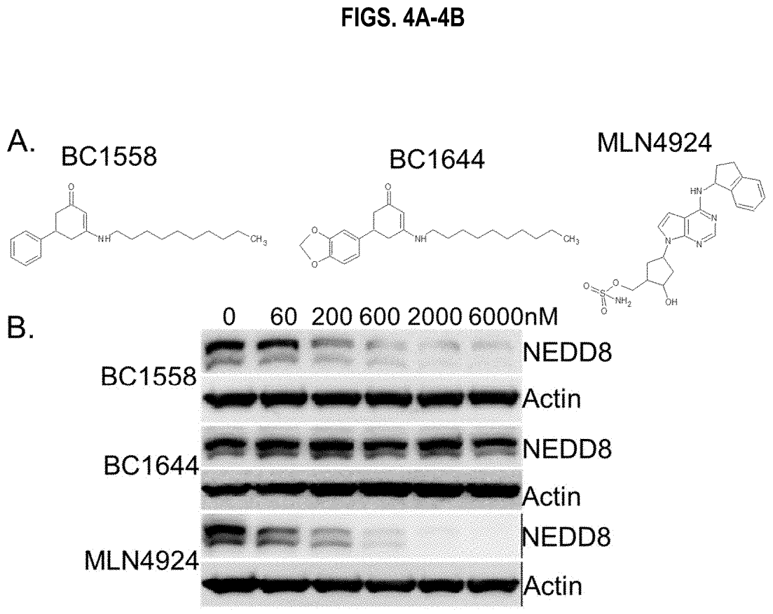

3-(decylamino)-5-phenylcyclohex-2-en-1-one was tested and compared with MLN4924 and showed comparable activity. 5-(1,3-benzodioxol-5-yl)-3-(decylamino)cyclohex-2-en-1-one was used as a negative control (FIG. 4A-B).

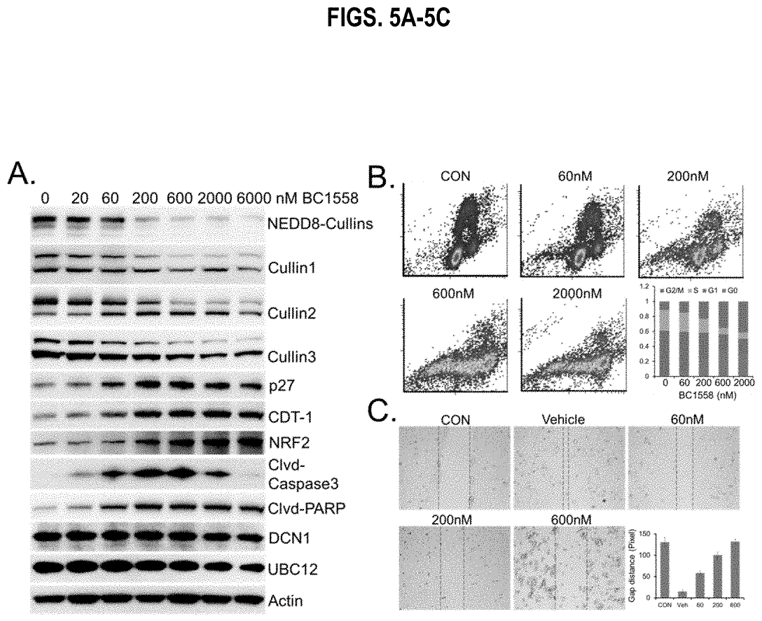

3-(decylamino)-5-phenylcyclohex-2-en-1-one was further tested in HepG2 cells in a dose dependent manner. As shown in FIG. 5A, BC1558 drastically reduced neddylation of several cullin isoforms. 3-(decylamino)-5-phenylcyclohex-2-en-1-one also effectively increased apoptosis markers Clvd-caspase3 and Clvd-PARP (FIG. 5A). 3-(decylamino)-5-phenylcyclohex-2-en-1-one also effectively decreased S phase, and increased G0 and G2/M phases in a dose dependent manner (FIG. 5B). Finally, 3-(decylamino)-5-phenylcyclohex-2-en-1-one was tested in a wound-healing assay and showed inhibition in hepG2 cells migration (FIG. 5C).

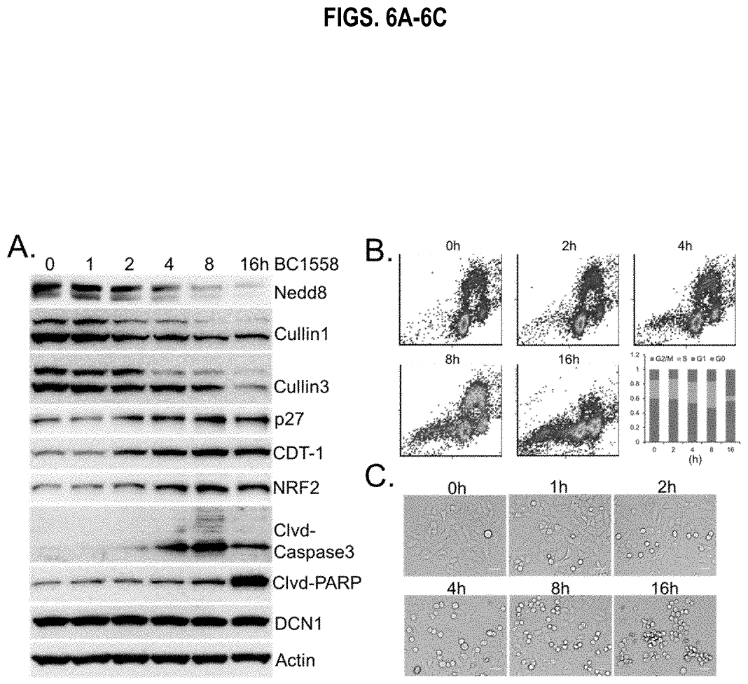

3-(decylamino)-5-phenylcyclohex-2-en-1-one was tested in HepG2 cells in a time dependent manner. As shown in FIG. 6A, 3-(decylamino)-5-phenylcyclohex-2-en-1-one drastically reduced neddylation of several cullin isoforms, but increased SCF substrates p27, NRF2 and CDT1. 3-(decylamino)-5-phenylcyclohex-2-en-1-one also effectively increased apoptosis markers Clvd-caspase3 and Clvd-PARP. At 16 h, 3-(decylamino)-5-phenylcyclohex-2-en-1-one also effectively decreased S phase, increased G0 and G2/M phases (FIG. 6B). Finally, 3-(decylamino)-5-phenylcyclohex-2-en-1-one treatment also resulted in accumulation of rounded, apoptotic hepG2 cells in a time dependent manner (FIG. 6C).

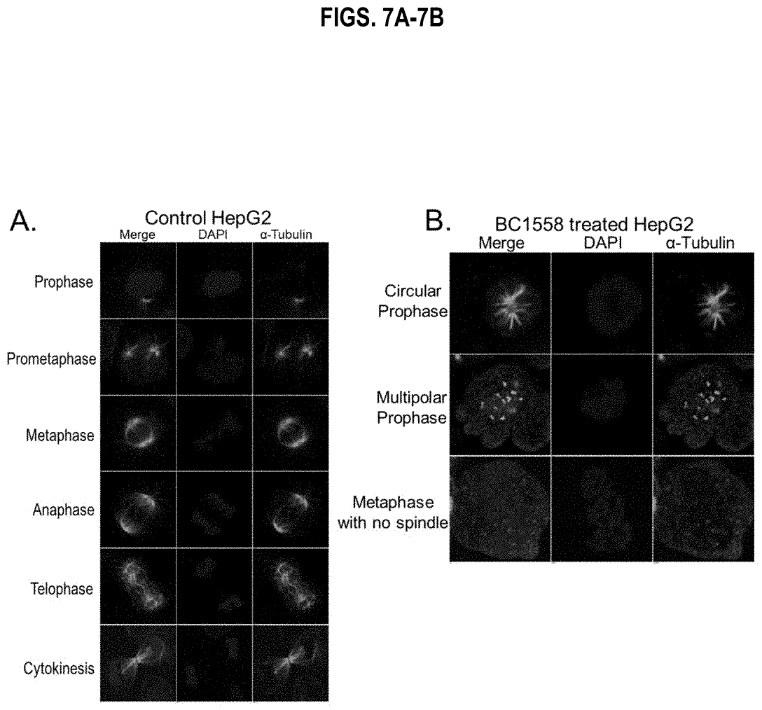

The integrity of the centrosome and mitotic spindles after 3-(decylamino)-5-phenylcyclohex-2-en-1-one treatment was examined. HepG2 cells were stained with antibody to α-tubulin to visualize the mitotic spindle (FIG. 7A-B). Abnormal cells with several representative mitotic abnormalities were also detected after 3-(decylamino)-5-phenylcyclohex-2-en-1-one treatment (FIG. 7B). The circular prophase configurations were observed (FIG. 7B, top panel). One classic phenotype of the circular chromosome configuration is circular figures arranged on monopolar spindles around large centrosomes. No spindles assembly in metaphase was observed (FIG. 7B, lower panel).

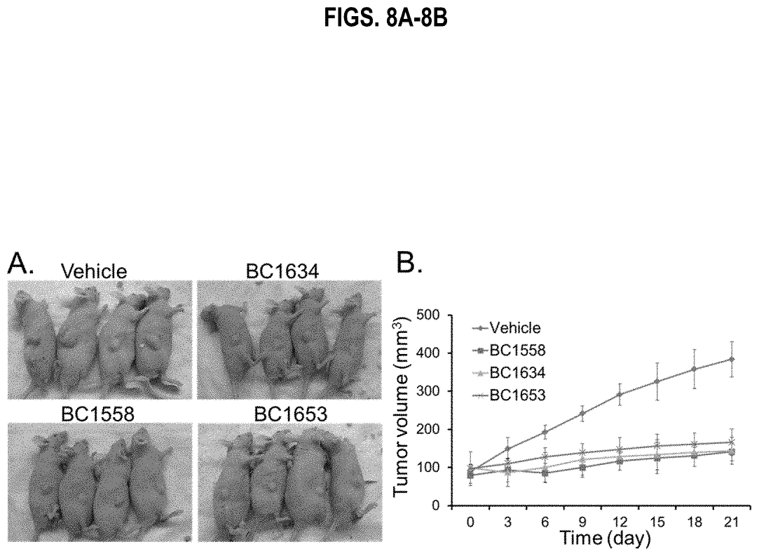

As a complementary in vivo model, tumorogenicity was further assessed after implanting HepG2 cells along with 3-(decylamino)-5-phenylcyclohex-2-en-1-one, 3-(dibenzylamino)-5-phenylcyclohex-2-en-1-one, 3-((4-phenoxybenzyl)amino)-5-phenylcyclohex-2-en-1-one treatment in athymic nude mice. Mice were implanted with HepG2 cells through subcutaneous injection. Two weeks later, once tumor size reached 0.1 cm³, the mice were randomized and the treatment began by adding compounds to the drinking water (15 mg/kg/d). Significantly reduced tumor size was observed with all compounds treatment compared to the vehicle (FIG. 8).

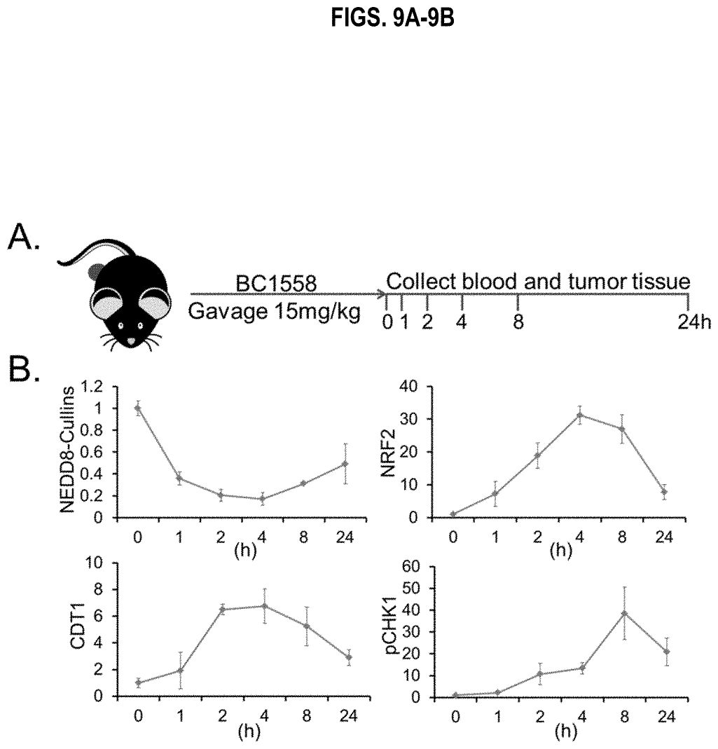

A PD/PK study was also performed. Mice were implanted with HepG2 cells (10⁶ cells) through subcutaneous injection. 5 weeks later, once tumor size reached 0.3 cm³, mice were randomized and given 1 dose of 3-(decylamino)-5-phenylcyclohex-2-en-1-one (15 mg/kg) by oral gavage (FIG. 9A). Protein immunoblotting was performed and showed that a single dose of 3-(decylamino)-5-phenylcyclohex-2-en-1-one resulted in a time-dependent decrease of NEDD8-cullin levels within 1 h after administration of compound with maximal effect 2-4 h post-dose (FIG. 9B). A single dose of 3-(decylamino)-5-phenylcyclohex-2-en-1-one also led to a time-dependent increase in the steady state levels of NRF2 and CDT1. NRF2 protein levels peaked 4 h after administration of BC1558 and started to decline by 8-24 h post-dose. The timing of CDT1 accumulation was compared to NRF2, peaking 4 h after 3-(decylamino)-5-phenylcyclohex-2-en-1-one administration. Evidence of DNA damage in the tumor was indicated by the increased levels of phosphorylated CHK1 (Ser 317) at 8 h after a single administration of 3-(decylamino)-5-phenylcyclohex-2-en-1-one.

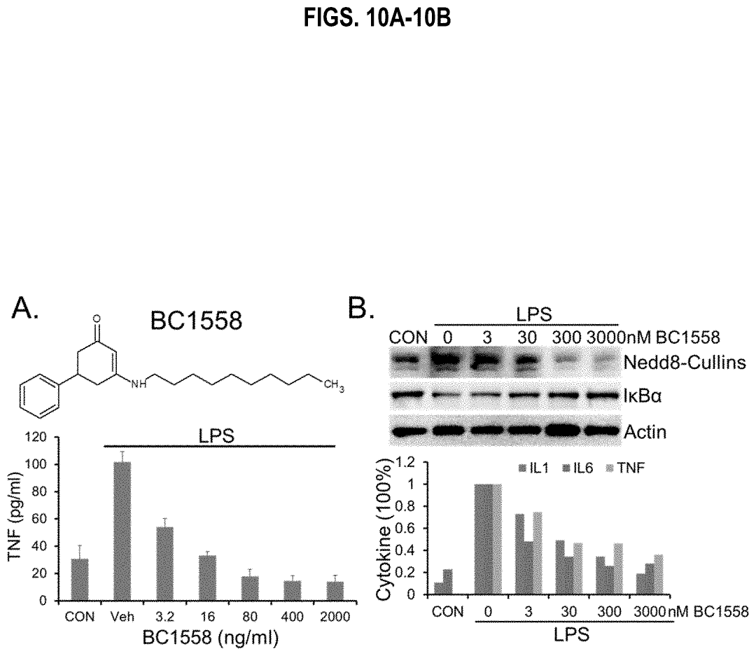

Since SCF machinery is believed to be essential for NF-kB activation, it was investigated whether the DCN1 inhibitors could affect inflammation. 3-(decylamino)-5-phenylcyclohex-2-en-1-one was first tested in PBMCs cells. Briefly, PBMC cells were treated with 3-(decylamino)-5-phenylcyclohex-2-en-1-one at various concentrations for 2 h before exposed to LPS (10 ng/ml) for 12 h. It was observed that 3-(decylamino)-5-phenylcyclohex-2-en-1-one potently inhibited LPS-induced TNF release from PBMC cells (FIG. 10A).

3-(decylamino)-5-phenylcyclohex-2-en-1-one was next tested in human lung tissue. Human lung slices were pretreated with 3-(decylamino)-5-phenylcyclohex-2-en-1-one for 18 h before LPS treatment (100 ng/ml) for additional 4 h. It was observed that 3-(decylamino)-5-phenylcyclohex-2-en-1-one potently inhibited LPS induced IL1, IL6 and TNF release from lung tissue. 3-(decylamino)-5-phenylcyclohex-2-en-1-one reduced nedd8-cullins level and rescued IκBα in a dose dependent manner.

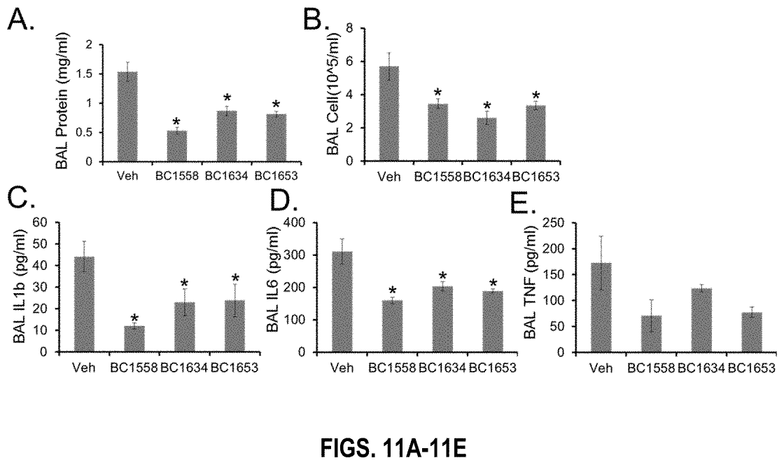

To assess in vivo anti-inflammatory activity of these compounds, they were tested using P. aeruginosa-induced pneumonia models. Briefly, C57BL/6J mice were administered i.t. with PA103 (1.5*104 CFU/mouse). Compounds were given through i.p. injection (30 mg/kg) at the same time. 18 h later mice were euthanized, and the lungs were lavaged with saline. DCN1 inhibitors significantly decreased lavage protein concentration and lavage cell counts in PA103 (FIG. 11A-B) stimulated mice. DCN1 inhibitors also significantly reduced lavage cytokine IL1 and IL6 (FIG. 11C-D).

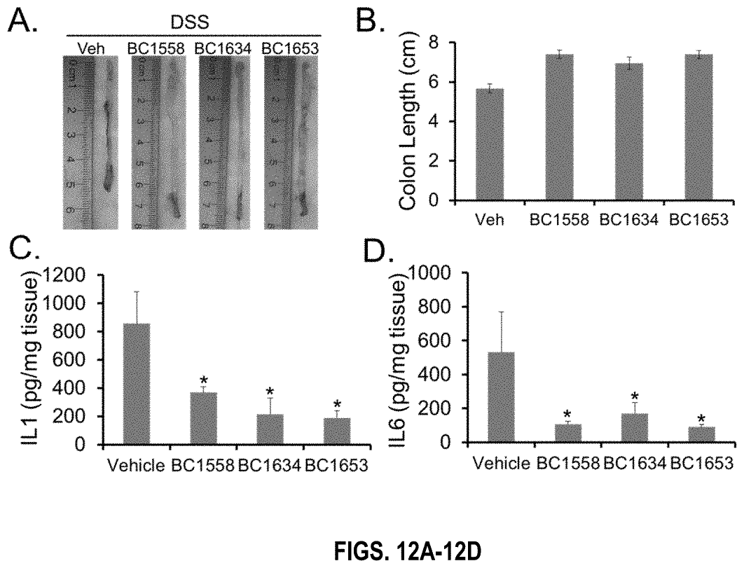

Also tested were 3-(decylamino)-5-phenylcyclohex-2-en-1-one (BC1558), 3-(dibenzylamino)-5-phenylcyclohex-2-en-1-one (BC1634) and 3-((4-phenoxybenzyl)amino)-5-phenylcyclohex-2-en-1-one (BC1653) in a mouse colitis model to confirm its anti-inflammatory activity. Briefly, C57BL6 mice were fed with water containing 3.5% ice were euthanized and colonic length was measured. DSS produced a significant decrease in colon length in mice, consistent with colonic inflammation (FIG. 12A-B). However, mice treated with DCN1 inhibitor showed significant increase in colon length compared vehicle. Colonic tissue cytokine levels were further analyzed. As shown in FIG. 12C-D, mice treated with DCN1 inhibitor showed a large reduction in IL1β and IL6 levels in colonic tissue compared to vehicle treated mice.

### VII. Additional Embodiments

The following additional embodiments are within the scope of this disclosure

### Embodiments A1-27

A1. A compound represented by formula (I) or formula (II):

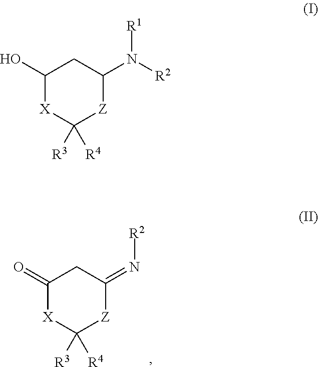

or a pharmaceutically acceptable salt, ester, hydrate or prodrug thereof, wherein:

X and Z are independently C₀₋₂-alkyl, —(CH₂)ₛ—NH—(CH₂)ₜ—, —(CH₂)ₛ—O—(CH₂)ₜ—, or —(CH₂)ₛ—C(NH₂)—(CH₂)ₜ—, wherein s and t are independently an integer of 0 or 1;

R¹ is selected from the group consisting of H, optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl;

R² is selected from the group consisting of optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl;

R³ is selected from the group consisting of H, optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl; and

R⁴ is selected from the group consisting of optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl.

A2. The compound of embodiment A1, wherein the compound is selected from a compound of formula (I) or a pharmaceutically acceptable salt, ester, hydrate or prodrug thereof.
A3. The compound of embodiment A1, wherein the compound is selected from a compound of formula (II) or a pharmaceutically acceptable salt, ester, hydrate or prodrug thereof.
A4. The compound of any of embodiments A1-A3, wherein R² is selected from the group consisting of C₁₋₁₆ alkyl, C₁₋₁₆ alkenyl, and C₁₋₁₆ alkynyl.
A5. The compound of any of embodiments A1-A4, wherein R² is selected from the group consisting of C₃₋₁₂ cycloalkyl, C₃₋₁₂ cycloalkyl-C₁₋₆alkyl, C₃₋₁₂ heterocycloalkyl, and C₃₋₁₂ heterocycloalkyl-C₁₋₆ alkyl.
A6. The compound of any of embodiments A1-A5, wherein R² is substituted with at least one of halogen, C₁₋₁₆ alkyl, C₁₋₁₆haloalkyl, hydroxyl, C₁₋₁₆ alkoxy, C₁₋₁₆haloalkoxy, amino, C₁₋₁₆alkylamino, or di-C₁₋₁₆alkylamino.
A7. The compound of any of embodiments A1-A6, wherein R⁴ is selected from the group consisting of 6-membered cycloalkyl, 6-membered heterocycloalkyl, 5-membered cycloalkyl, and 5-membered heterocycloalkyl.
A8. The compound of any of embodiments A1-A7, wherein R⁴ is selected from the group consisting of pyrimidine and pyridine.
A9. The compound of any of embodiments A1-A8, wherein R⁴ is substituted with at least one substituent selected from the group consisting of halogen, C₁₋₆ alkyl, C₁₋₆ haloalkyl, hydroxyl, C₁₋₆ alkoxy, C₁₋₆ haloalkoxy, amino, C₁₋₆ alkylamino, and di-C₁₋₆ alkylamino.
A10. The compound of any of embodiments A1-A9, wherein R⁴ is selected from the group consisting of: C₁₋₁₈ alkyl, alkenyl or alkynyl; C₀₋₁₈ alkyl, alkenyl or alkynyl-C₆₋₁₄ aryl; C₀₋₁₈ alkyl, alkenyl or alkynyl-C₃₋₁₂ cycloalkyl; C₀₋₁₈ alkyl, alkenyl or alkynyl-C₃₋₁₂ cycloalkyl-C₆₋₁₄ aryl; C₀₋₁₈ alkyl, alkenyl or alkynyl-di-C₆₋₁₄ aryl; C₀₋₁₈ alkyl, alkenyl or alkynyl-C₆₋₁₄ aryl-C₆₋₁₄ aryl; C₀₋₁₈ alkyl, alkenyl or alkynyl-C₆₋₁₄ aryl-O—C₆₋₁₄ aryl; and C₀₋₁₈ alkyl, alkenyl or alkynyl-C₆₋₁₄ aryl-C₂₋₅ N or S-heteroaryl.
A11. The compound of any of embodiments A1-A10, wherein R⁴ is selected from the group consisting of: C₆₋₁₄ n-alkyl; C₁₋₆ n-alkyl-phenyl, optionally substituted with a phenyl, C₁₋₃-alkyl-phenyl and —O-phenyl.
A12. The compound of any of embodiments A1-A11, wherein R³ is H.
A13. The compound of any of embodiments A1-A12, wherein R³ is selected from the group consisting of: C₁₋₁₈ alkyl, alkenyl or alkynyl; C₀₋₁₈ alkyl, alkenyl or alkynyl-C₆₋₁₄ aryl; C₀₋₁₈ alkyl, alkenyl or alkynyl-C₃₋₁₂ cycloalkyl; C₀₋₁₈ alkyl, alkenyl or alkynyl-C₃₋₁₂ cycloalkyl-C₆₋₁₄ aryl; C₀₋₁₈ alkyl, alkenyl or alkynyl-di-C₆₋₁₄ aryl; C₀₋₁₈ alkyl, alkenyl or alkynyl-C₆₋₁₄ aryl-C₆₋₁₄ aryl; C₀₋₁₈ alkyl, alkenyl or alkynyl-C₆₋₁₄ aryl-O—C₆₋₁₄ aryl; and C₀₋₁₈ alkyl, alkenyl or alkynyl-C₆₋₁₄ aryl-C₂₋₅ N, O or S-heteroaryl.
A14. The compound of any of embodiments A1-A13, wherein R¹ is H.
A15. The compound of any of embodiments A1-A14, wherein R² is selected from the group consisting of: C₁₋₁₈ alkyl, alkenyl or alkynyl; C₀₋₁₈ alkyl, alkenyl or alkynyl-C₆₋₁₄ aryl; C₀₋₁₈ alkyl, alkenyl or alkynyl-C₃₋₁₂ cycloalkyl; C₀₋₁₈ alkyl, alkenyl or alkynyl-C₃₋₁₂ cycloalkyl-C₆₋₁₄ aryl; C₀₋₁₈ alkyl, alkenyl or alkynyl-di-C₆₋₁₄ aryl; C₀₋₁₈ alkyl, alkenyl or alkynyl-C₆₋₁₄ aryl-C₆₋₁₄ aryl; C₀₋₁₈ alkyl, alkenyl or alkynyl-C₆₋₁₄ aryl-O—C₆₋₁₄ aryl; and C₀₋₁₈ alkyl, alkenyl or alkynyl-C₆₋₁₄ aryl-C₂₋₅ N, O or S-heteroaryl.
A16. The compound of any of embodiments A1-A15, wherein X and Z are CH₂.
A17. A compound selected from the following shown in Table 4:

TABLE 4

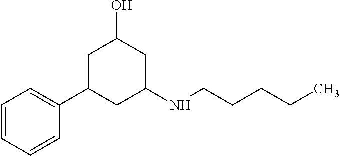 [SMILES: CCCCCNC1CC(O)CC(c2ccccc2)C1]

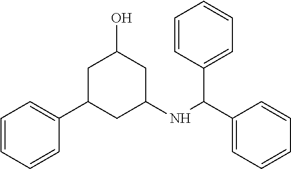 [SMILES: OC1CC(NC(c2ccccc2)c2ccccc2)CC(c2ccccc2)C1]

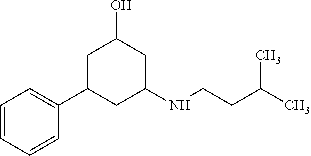 [SMILES: CC(C)CCNC1CC(O)CC(c2ccccc2)C1]

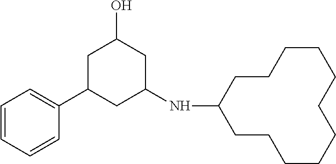 [SMILES: OC1CC(NC2CCCCCCCCCCC2)CC(c2ccccc2)C1]

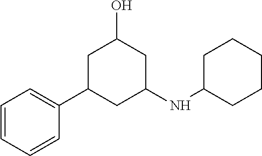 [SMILES: OC1CC(NC2CCCCC2)CC(c2ccccc2)C1]

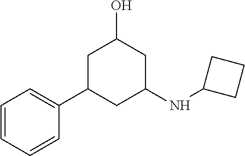 [SMILES: OC1CC(NC2CCC2)CC(c2ccccc2)C1]

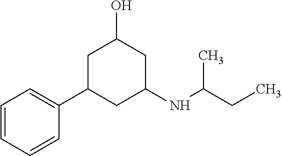 [SMILES: CCC(C)NC1CC(O)CC(c2ccccc2)C1]

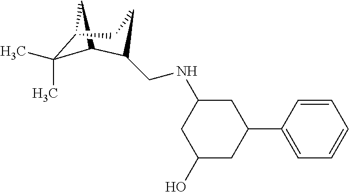

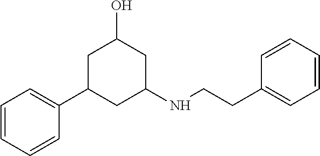 [SMILES: OC1CC(NCCc2ccccc2)CC(c2ccccc2)C1]

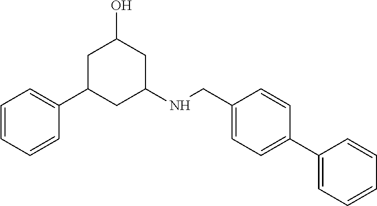 [SMILES: OC1CC(NCc2ccc(-c3ccccc3)cc2)CC(c2ccccc2)C1]

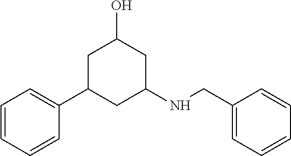 [SMILES: OC1CC(NCc2ccccc2)CC(c2ccccc2)C1]

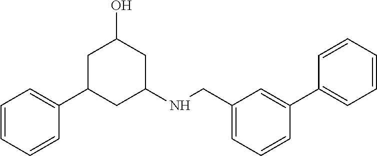 [SMILES: OC1CC(NCc2cccc(-c3ccccc3)c2)CC(c2ccccc2)C1]

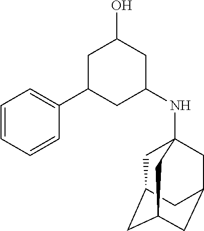 [SMILES: OC1CC(NC23CC4C[C@H](C2)C[C@@H](C4)C3)CC(c2ccccc2)C1]

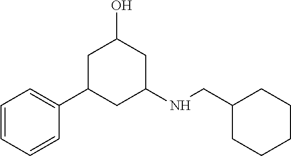 [SMILES: OC1CC(NCC2CCCCC2)CC(c2ccccc2)C1]

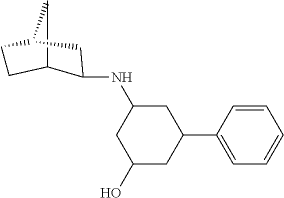

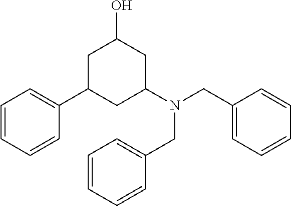 [SMILES: OC1CC(c2ccccc2)CC(N(Cc2ccccc2)Cc2ccccc2)C1]

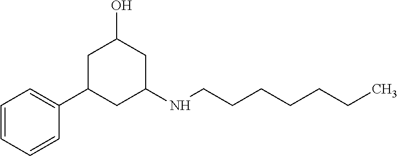 [SMILES: CCCCCCCNC1CC(O)CC(c2ccccc2)C1]

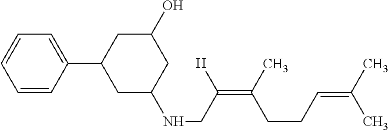 [SMILES: CC(C)=CCC/C(C)=C\CNC1CC(O)CC(c2ccccc2)C1]

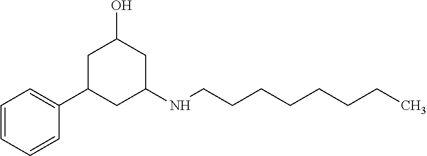 [SMILES: CCCCCCCCNC1CC(O)CC(c2ccccc2)C1]

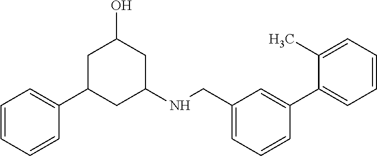 [SMILES: Cc1ccccc1-c1cccc(CNC2CC(O)CC(c3ccccc3)C2)c1]

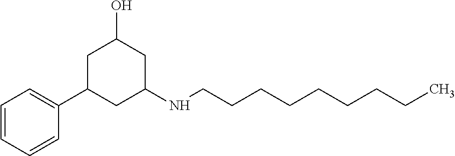 [SMILES: CCCCCCCCCNC1CC(O)CC(c2ccccc2)C1]

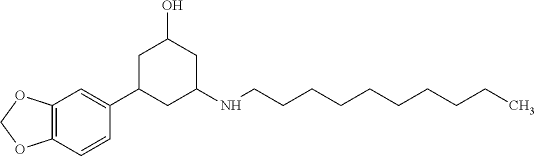 [SMILES: CCCCCCCCCCNC1CC(O)CC(c2ccc3c(c2)OCO3)C1]

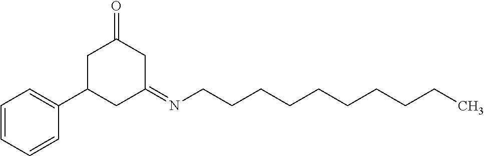 [SMILES: CCCCCCCCCC/N=C1\CC(=O)CC(c2ccccc2)C1]

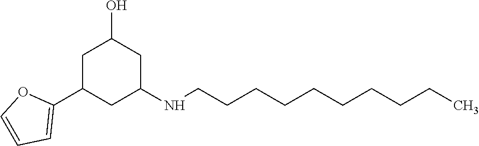 [SMILES: CCCCCCCCCCNC1CC(O)CC(c2ccco2)C1]

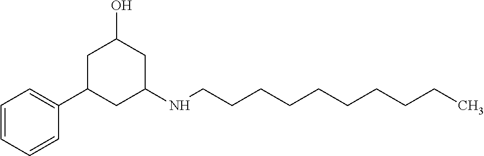 [SMILES: CCCCCCCCCCNC1CC(O)CC(c2ccccc2)C1]

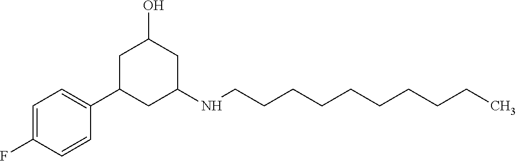 [SMILES: CCCCCCCCCCNC1CC(O)CC(c2ccc(F)cc2)C1]

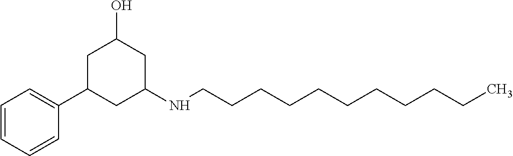 [SMILES: CCCCCCCCCCCNC1CC(O)CC(c2ccccc2)C1]

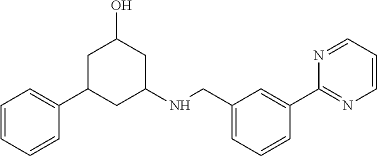 [SMILES: OC1CC(NCc2cccc(-c3ncccn3)c2)CC(c2ccccc2)C1]

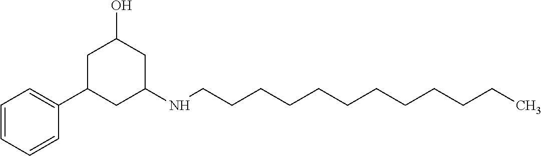 [SMILES: CCCCCCCCCCCCNC1CC(O)CC(c2ccccc2)C1]

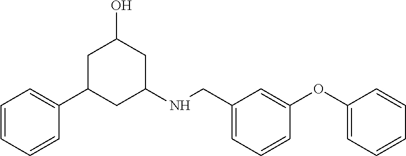 [SMILES: OC1CC(NCc2cccc(Oc3ccccc3)c2)CC(c2ccccc2)C1]

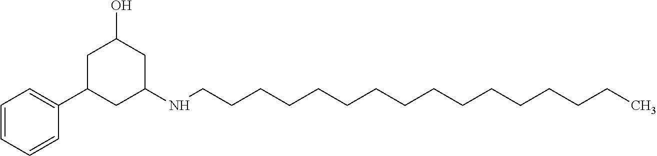 [SMILES: CCCCCCCCCCCCCCCCNC1CC(O)CC(c2ccccc2)C1]

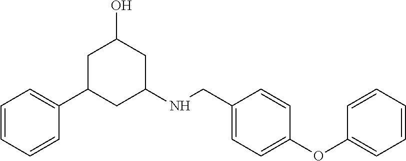 [SMILES: OC1CC(NCc2ccc(Oc3ccccc3)cc2)CC(c2ccccc2)C1]

A18. A pharmaceutical composition comprising a compound of any of embodiments A1-A17 and a pharmaceutically acceptable excipient.
A19. A method of treating an acute and chronic inflammation disorder in a patient in need thereof, the method comprising administering to the patient an effective amount of a compound or composition according to any of embodiments A1-A18
A20. The method of embodiment A19, wherein the acute and chronic inflammation disorder is selected from the group consisting of asthma, chronic obstructive lung disease, pulmonary fibrosis, pneumonitis (including hypersensitivity pneumonitis and radiation pneumonitis), pneumonia, cystic fibrosis, psoriasis, arthritis/rheumatoid arthritis, rhinitis, pharyngitis, cystitis, prostatitis, dermatitis, allergy including hayfever, nephritis, conjunctivitis, encephalitis, meningitis, opthalmitis, uveitis, pleuritis, pericarditis, myocarditis, atherosclerosis, human immunodeficiency virus related inflammation, diabetes, osteoarthritis, psoriatic arthritis, inflammatory bowel disease (Crohn's disease, ulcerative colitis)/colitis, sepsis, vasculitis, bursitis, connective tissue disease, autoimmune diseases such as systemic lupus erythematosis (SLE), polymyalgia rheumatica, scleroderma, Wegener's granulomatosis, temporal arteritis, vasculitis, cryoglobulinemia, and multiple sclerosis, viral or influenza-induced inflammation, and edema.
A21. The method of embodiment A19 or A20, wherein the effective amount is from about 0.5 mg to about 500 mg of a compound of any of the preceding embodiments.
A22. The method of any one of embodiment A19-21, wherein the effective amount is about 0.5 mg/kg to about 500 mg/kg of compound per kg of patient body weight.
A23. The method of any one of embodiment A19-22, wherein the administration is oral administration, administration via implants, parenteral injection, intravenous injection, intraperitoneal injection, subcutaneous injection, bolus injection, infusion, rectal administration, vaginal administration, transdermal administration, inhalation, or any combination thereof.
A24. The method of any one of embodiment A19-23, wherein the method further includes a step of administrating at least one anti-inflammatory agents, antimicrobial agents, matrix metalloprotease inhibitors, lipoxygenase inhibitors, cytokine antagonists, immunosuppressants, anti-cancer agents, anti-viral agents, cytokines, growth factors, immunomodulators, prostaglandins, anti-vascular hyperproliferation compounds, and combinations thereof either concurrently with the compound or composition according to any one of embodiments A1-18 or in the same course of treatment.
A25. The method of any one of embodiment A19-24, wherein the method is for treating sepsis, pneumonia, influenza-induced inflammation, edema, neuropathy, colitis, arthritis, Crohn's disease, diabetes, skin, eye and ear inflammation (e.g., psoriasis, uveitis/opthalmitis, external otitis), systemic lupus erythematosis (SLE), or systemic lupus erythematosis (SLE) in the patient in need thereof.
A26. Else of a compound or composition according to any one of embodiment A1-18 for treating acute and chronic inflammation disorders such as asthma, chronic obstructive lung disease, pulmonary fibrosis, pneumonitis (including hypersensitivity pneumonitis and radiation pneumonitis), pneumonia, cystic fibrosis, psoriasis, arthritis/rheumatoid arthritis, rhinitis, pharyngitis, cystitis, prostatitis, dermatitis, allergy including hayfever, nephritis, conjunctivitis, encephalitis, meningitis, opthalmitis, uveitis, pleuritis, pericarditis, myocarditis, atherosclerosis, human immunodeficiency virus related inflammation, diabetes, osteoarthritis, psoriatic arthritis, inflammatory bowel disease (Crohn's disease, ulcerative colitis)/colitis, sepsis, vasculitis, bursitis, connective tissue disease, autoimmune diseases such as systemic lupus erythematosis (SLE), polymyalgia rheumatica, scleroderma, Wegener's granulomatosis, temporal arteritis, vasculitis, cryoglobulinemia, and multiple sclerosis, viral or influenza-induced inflammation, or edema.

A27. Use of a compound or composition according to any one of embodiment A1-18 in the manufacture of a medicament for use in treating acute and chronic inflammation disorders such as asthma, chronic obstructive lung disease, pulmonary fibrosis, pneumonitis (including hypersensitivity pneumonitis and radiation pneumonitis), pneumonia, cystic fibrosis, psoriasis, arthritis/rheumatoid arthritis, rhinitis, pharyngitis, cystitis, prostatitis, dermatitis, allergy including hayfever, nephritis, conjunctivitis, encephalitis, meningitis, opthalmitis, uveitis, pleuritis, pericarditis, myocarditis, atherosclerosis, human immunodeficiency virus related inflammation, diabetes, osteoarthritis, psoriatic arthritis, inflammatory bowel disease (Crohn's disease, ulcerative colitis)/colitis, sepsis, vasculitis, bursitis, connective tissue disease, autoimmune diseases such as systemic lupus erythematosis (SLE), polymyalgia rheumatica, scleroderma, Wegener's granulomatosis, temporal arteritis, vasculitis, cryoglobulinemia, and multiple sclerosis, viral or influenza-induced inflammation, or edema.

### Embodiments B1-28

B1. A compound represented by formula (I):

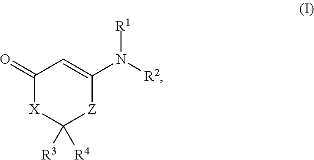

or a pharmaceutically acceptable salt, ester, hydrate or prodrug thereof, wherein:
X and Z are independently C₀₋₂-alkyl, —(CH₂)ₛ—NH—(CH₂)ₜ—, —(CH₂)ₛ—O—(CH₂)ₜ—, or —(CH₂)ₛ—C(NH₂)—(CH₂)ₜ—, wherein s and t are independently an integer of 0 or 1;
R¹ is selected from the group consisting of H, optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl;
R² is selected from the group consisting of optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl;
R³ is selected from the group consisting of H, optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl; and
R⁴ is selected from the group consisting of optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl.
B2. The compound of embodiment B1, wherein the compound is selected from a compound of formula (I) or a pharmaceutically acceptable salt, ester, hydrate or prodrug thereof.
B3. The compound of embodiment B1, wherein the compound is selected from a pharmaceutically acceptable salt of compound of formula (I).
B4. The compound of any of embodiments B1-B3, wherein R² is selected from the group consisting of C₁₋₁₆ alkyl, C₁₋₁₆ alkenyl, and C₁₋₁₆ alkynyl.
B5. The compound of any of embodiments B1-B4, wherein R² is selected from the group consisting of C₃₋₁₂ cycloalkyl, C₃₋₁₂ cycloalkyl-C₁₋₆alkyl, C₃₋₁₂ heterocycloalkyl, and C₃₋₁₂ heterocycloalkyl-C₁₋₆ alkyl.
B6. The compound of any of embodiments B1-B5, wherein R² is substituted with at least one of halogen, C₁₋₁₆ alkyl, C₁₋₁₆haloalkyl, hydroxyl, C₁₋₁₆ alkoxy, C₁₋₁₆haloalkoxy, amino, C₁₋₁₆alkylamino, or di-C₁₋₁₆alkylamino.
B7. The compound of any of embodiments B1-B6, wherein R⁴ is selected from the group consisting of 6-membered cycloalkyl, 6-membered heterocycloalkyl, 5-membered cycloalkyl, and 5-membered heterocycloalkyl.
B8. The compound of any of the preceding embodiments, wherein R⁴ is selected from the group consisting of pyrimidine and pyridine.
B9. The compound of any of embodiments B1-B8, wherein R⁴ is substituted with at least one substituent selected from the group consisting of halogen, C₁₋₆ alkyl, C₁₋₆ haloalkyl, hydroxyl, C₁₋₆ alkoxy, C₁₋₆ haloalkoxy, amino, C₁₋₆ alkylamino, and di-C₁₋₆ alkylamino.
B10. The compound of any of embodiments B1-B9, wherein R⁴ is selected from the group consisting of: C₁₋₁₈ alkyl, alkenyl or alkynyl; C₀₋₁₈ alkyl, alkenyl or alkynyl-C₆₋₁₄ aryl; C₀₋₁₈ alkyl, alkenyl or alkynyl-C₃₋₁₂ cycloalkyl; C₀₋₁₈ alkyl, alkenyl or alkynyl-C₃₋₁₂ cycloalkyl-C₆₋₁₄ aryl; C₀₋₁₈ alkyl, alkenyl or alkynyl-di-C₆₋₁₄ aryl; C₀₋₁₈ alkyl, alkenyl or alkynyl-C₆₋₁₄ aryl-C₆₋₁₄ aryl; C₀₋₁₈ alkyl, alkenyl or alkynyl-C₆₋₁₄ aryl-O—C₆₋₁₄ aryl; and C₀₋₁₈ alkyl, alkenyl or alkynyl-C₆₋₁₄ aryl-C₂₋₅ N or S-heteroaryl.
B11. The compound of any of embodiments B1-B10, wherein R⁴ is selected from the group consisting of: C₆₋₁₄ n-alkyl; C₁₋₆ n-alkyl-phenyl, optionally substituted with a phenyl, C₁₋₃-alkyl-phenyl and —O-phenyl.
B12. The compound of any of embodiments B1-B11, wherein R³ is H.
B13. The compound of any of embodiments B1-B12, wherein R³ is selected from the group consisting of: C₁₋₁₈ alkyl, alkenyl or alkynyl; C₀₋₁₈ alkyl, alkenyl or alkynyl-C₆₋₁₄ aryl; C₀₋₁₈ alkyl, alkenyl or alkynyl-C₃₋₁₂ cycloalkyl; C₀₋₁₈ alkyl, alkenyl or alkynyl-C₃₋₁₂ cycloalkyl-C₆₋₁₄ aryl; C₀₋₁₈ alkyl, alkenyl or alkynyl-di-C₆₋₁₄ aryl; C₀₋₁₈ alkyl, alkenyl or alkynyl-C₆₋₁₄ aryl-C₆₋₁₄ aryl; C₀₋₁₈ alkyl, alkenyl or alkynyl-C₆₋₁₄ aryl-O—C₆₋₁₄ aryl; and C₀₋₁₈ alkyl, alkenyl or alkynyl-C₆₋₁₄ aryl-C₂₋₅ N, O or S-heteroaryl.
B14. The compound of any of embodiments B1-B13, wherein R¹ is H.
B15. The compound of any of embodiments B1-B14, wherein R² is selected from the group consisting of: C₁₋₁₈ alkyl, alkenyl or alkynyl; C₀₋₁₈ alkyl, alkenyl or alkynyl-C₆₋₁₄ aryl; C₀₋₁₈ alkyl, alkenyl or alkynyl-C₃₋₁₂ cycloalkyl; C₀₋₁₈ alkyl, alkenyl or alkynyl-C₃₋₁₂ cycloalkyl-C₆₋₁₄ aryl; C₀₋₁₈ alkyl, alkenyl or alkynyl-di-C₆₋₁₄ aryl; C₀₋₁₈ alkyl, alkenyl or alkynyl-C₆₋₁₄ aryl-C₆₋₁₄ aryl; C₀₋₁₈ alkyl, alkenyl or alkynyl-C₆₋₁₄ aryl-O—C₆₋₁₄ aryl; and C₀₋₁₈ alkyl, alkenyl or alkynyl-C₆₋₁₄ aryl-C₂₋₅ N, O or S-heteroaryl.
B16. The compound of any of embodiments B1-B15, wherein X and Z are CH₂.
B17. A compound selected from the following shown in Table 5:

TABLE 5

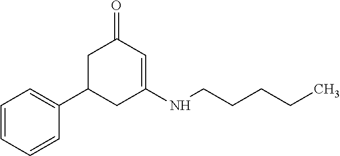 [SMILES: CCCCCNC1=CC(=O)CC(c2ccccc2)C1]

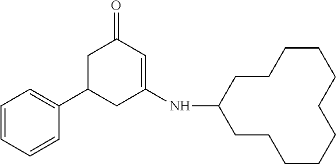 [SMILES: O=C1C=C(NC2CCCCCCCCCCC2)CC(c2ccccc2)C1]

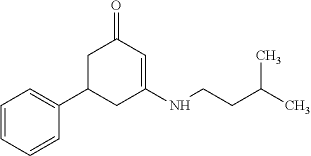 [SMILES: CC(C)CCNC1=CC(=O)CC(c2ccccc2)C1]

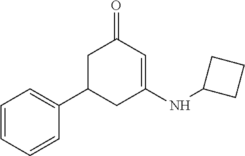 [SMILES: O=C1C=C(NC2CCC2)CC(c2ccccc2)C1]

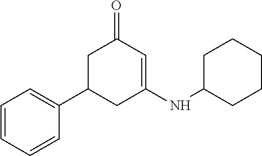 [SMILES: O=C1C=C(NC2CCCCC2)CC(c2ccccc2)C1]

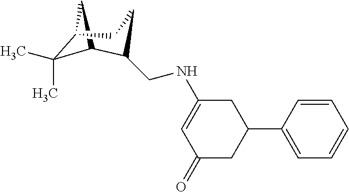

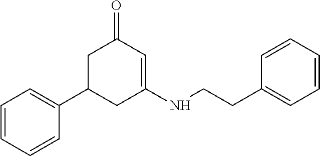 [SMILES: O=C1C=C(NCCc2ccccc2)CC(c2ccccc2)C1]

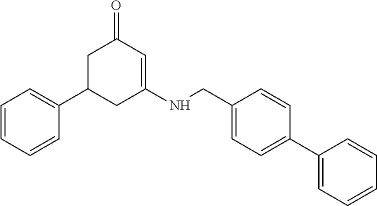 [SMILES: O=C1C=C(NCc2ccc(-c3ccccc3)cc2)CC(c2ccccc2)C1]

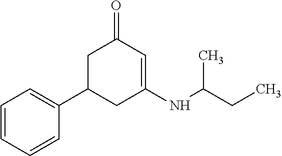 [SMILES: CCC(C)NC1=CC(=O)CC(c2ccccc2)C1]

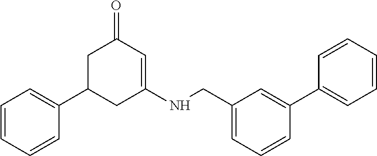 [SMILES: O=C1C=C(NCc2cccc(-c3ccccc3)c2)CC(c2ccccc2)C1]

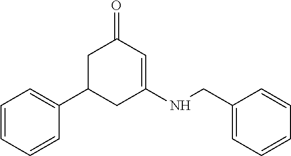 [SMILES: O=C1C=C(NCc2ccccc2)CC(c2ccccc2)C1]

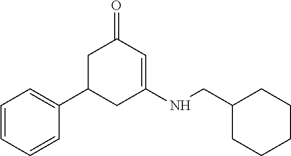 [SMILES: O=C1C=C(NCC2CCCCC2)CC(c2ccccc2)C1]

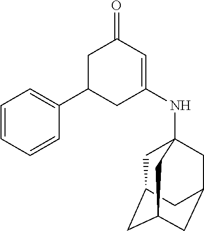 [SMILES: O=C1C=C(NC23CC4C[C@H](C2)C[C@@H](C4)C3)CC(c2ccccc2)C1]

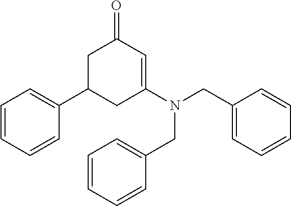 [SMILES: O=C1C=C(N(Cc2ccccc2)Cc2ccccc2)CC(c2ccccc2)C1]

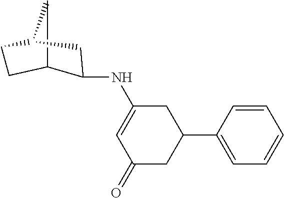

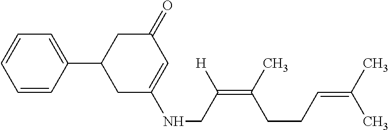 [SMILES: CC(C)=CCC/C(C)=C\CNC1=CC(=O)CC(c2ccccc2)C1]

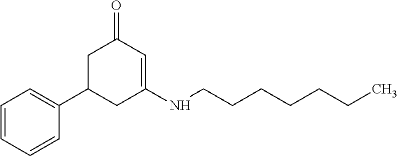 [SMILES: CCCCCCCNC1=CC(=O)CC(c2ccccc2)C1]

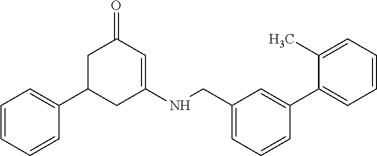 [SMILES: Cc1ccccc1-c1cccc(CNC2=CC(=O)CC(c3ccccc3)C2)c1]

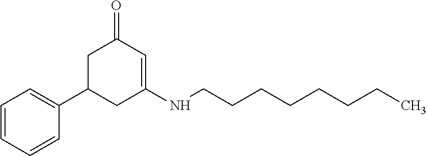 [SMILES: CCCCCCCCNC1=CC(=O)CC(c2ccccc2)C1]

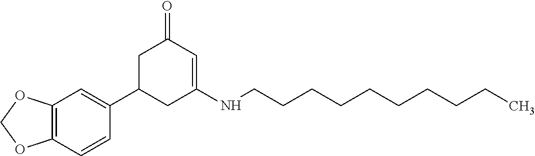 [SMILES: CCCCCCCCCCNC1=CC(=O)CC(c2ccc3c(c2)OCO3)C1]

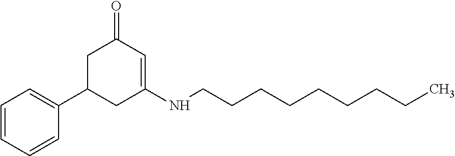 [SMILES: CCCCCCCCCNC1=CC(=O)CC(c2ccccc2)C1]

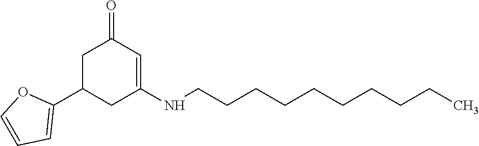 [SMILES: CCCCCCCCCCNC1=CC(=O)CC(c2ccco2)C1]

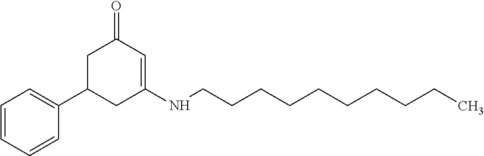 [SMILES: CCCCCCCCCCNC1=CC(=O)CC(c2ccccc2)C1]

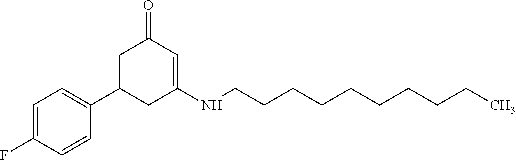 [SMILES: CCCCCCCCCCNC1=CC(=O)CC(c2ccc(F)cc2)C1]

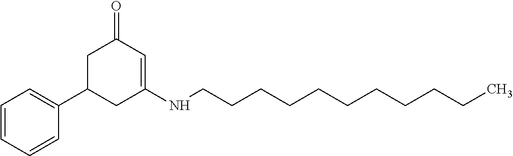 [SMILES: CCCCCCCCCCCNC1=CC(=O)CC(c2ccccc2)C1]

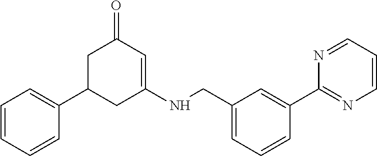 [SMILES: O=C1C=C(NCc2cccc(-c3ncccn3)c2)CC(c2ccccc2)C1]

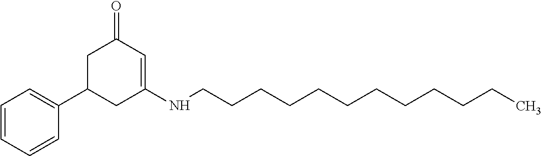 [SMILES: CCCCCCCCCCCCNC1=CC(=O)CC(c2ccccc2)C1]

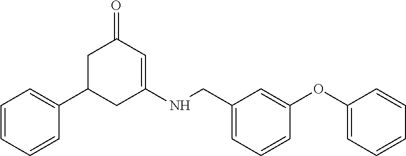 [SMILES: O=C1C=C(NCc2cccc(Oc3ccccc3)c2)CC(c2ccccc2)C1]

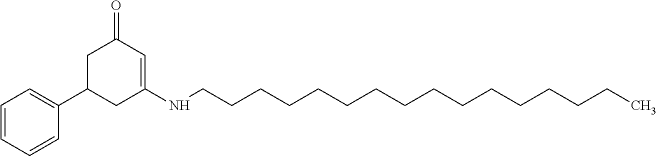 [SMILES: CCCCCCCCCCCCCCCCNC1=CC(=O)CC(c2ccccc2)C1]

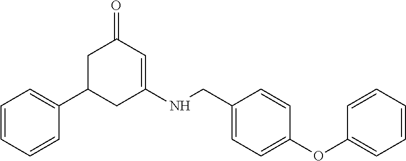 [SMILES: O=C1C=C(NCc2ccc(Oc3ccccc3)cc2)CC(c2ccccc2)C1]

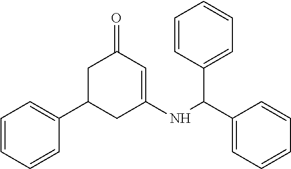 [SMILES: O=C1C=C(NC(c2ccccc2)c2ccccc2)CC(c2ccccc2)C1]

B18. A pharmaceutical composition comprising a compound of any of embodiments B1-B17 and a pharmaceutically acceptable excipient.
B19. A method of treating cancer in a patient in need thereof, the method comprising administering to the patient an effective amount of a compound of any of embodiments B1-B17 or a composition of embodiment B18, preferably the cancer being selected from brain cancer, throat cancer, thyroid cancer, esophagus cancer, tonsil cancer, lung cancer, prostate cancer, colorectal cancer, stomach cancer, liver cancer, pancreatic cancer, gallbladder cancer, bladder cancer, rectal cancer, testicle cancer, breast cancer, cervical cancer, ovarian cancer, skin cancer, melanoma, leukemia, lymphoma, and multiple myeloma.
B20. A method of treating an acute and chronic inflammation disorder in a patient in need thereof, the method comprising administering to the patient an effective amount of a compound of any of embodiments B1-B17 or a composition of embodiment B18.
B21. The method of embodiment B20, wherein the acute and chronic inflammation disorder is selected from the group consisting of asthma, chronic obstructive lung disease, pulmonary fibrosis, pneumonitis (including hypersensitivity pneumonitis and radiation pneumonitis), pneumonia, cystic fibrosis, psoriasis, arthritis/rheumatoid arthritis, rhinitis, pharyngitis, cystitis, prostatitis, dermatitis, allergy including hayfever, nephritis, conjunctivitis, encephalitis, meningitis, opthalmitis, uveitis, pleuritis, pericarditis, myocarditis, atherosclerosis, human immunodeficiency virus related inflammation, diabetes, osteoarthritis, psoriatic arthritis, inflammatory bowel disease (Crohn's disease, ulcerative colitis)/colitis, sepsis, vasculitis, bursitis, connective tissue disease, autoimmune diseases such as systemic lupus erythematosis (SLE), polymyalgia rheumatica, scleroderma, Wegener's granulomatosis, temporal arteritis, vasculitis, cryoglobulinemia, and multiple sclerosis, viral or influenza-induced inflammation, and edema.
B22. The method of embodiment B20 or 21, wherein the effective amount is from about 0.5 mg to about 500 mg of a compound of any of the preceding embodiments.
B23. The method of any one of embodiments B19-22, wherein the effective amount is about 0.5 mg/kg to about 500 mg/kg of compound per kg of patient body weight.
B24. The method of any one of embodiments B19-23, wherein the administration is oral administration, administration via implants, parenteral injection, intravenous injection, intraperitoneal injection, subcutaneous injection, bolus injection, infusion, rectal administration, vaginal administration, transdermal administration, inhalation, or any combination thereof.
B25. The method of any one of embodiments B19-24, wherein the method further includes a step of administrating at least one anti-inflammatory agents, antimicrobial agents, matrix metalloprotease inhibitors, lipoxygenase inhibitors, cytokine antagonists, immunosuppressants, anti-cancer agents, anti-viral agents, cytokines, growth factors, immunomodulators, prostaglandins, anti-vascular hyperproliferation compounds, and combinations thereof either concurrently with the compound or composition according to any one of embodiments B1-18 or in the same course of treatment.
B26. The method of any one of embodiments B19-25, wherein the method is for treating sepsis, pneumonia, influenza-induced inflammation, edema, neuropathy, colitis, arthritis, Crohn's disease, diabetes, skin, eye and ear inflammation (e.g., psoriasis, uveitis/opthalmitis, external otitis), systemic lupus erythematosis (SLE), or systemic lupus erythematosis (SLE) in the patient in need thereof.
B27. Else of a compound or composition according to any one of embodiments B1-18 for treating acute and chronic inflammation disorders such as asthma, chronic obstructive lung disease, pulmonary fibrosis, pneumonitis (including hypersensitivity pneumonitis and radiation pneumonitis), pneumonia, cystic fibrosis, psoriasis, arthritis/rheumatoid arthritis, rhinitis, pharyngitis, cystitis, prostatitis, dermatitis, allergy including hayfever, nephritis, conjunctivitis, encephalitis, meningitis, opthalmitis, uveitis, pleuritis, pericarditis, myocarditis, atherosclerosis, human immunodeficiency virus related inflammation, diabetes, osteoarthritis, psoriatic arthritis, inflammatory bowel disease (Crohn's disease, ulcerative colitis)/colitis, sepsis, vasculitis, bursitis, connective tissue disease, autoimmune diseases such as systemic lupus erythematosis (SLE), polymyalgia rheumatica, scleroderma, Wegener's granulomatosis, temporal arteritis, vasculitis, cryoglobulinemia, and multiple sclerosis, viral or influenza-induced inflammation, or edema.
B28. Use of a compound or composition according to any one of embodiments B1-18 in the manufacture of a medicament for use in treating acute and chronic inflammation disorders such as asthma, chronic obstructive lung disease, pulmonary fibrosis, pneumonitis (including hypersensitivity pneumonitis and radiation pneumonitis), pneumonia, cystic fibrosis, psoriasis, arthritis/rheumatoid arthritis, rhinitis, pharyngitis, cystitis, prostatitis, dermatitis, allergy including hayfever, nephritis, conjunctivitis, encephalitis, meningitis, opthalmitis, uveitis, pleuritis, pericarditis, myocarditis, atherosclerosis, human immunodeficiency virus related inflammation, diabetes, osteoarthritis, psoriatic arthritis, inflammatory bowel disease (Crohn's disease, ulcerative colitis)/colitis, sepsis, vasculitis, bursitis, connective tissue disease, autoimmune diseases such as systemic lupus erythematosis (SLE), polymyalgia rheumatica, scleroderma, Wegener's granulomatosis, temporal arteritis, vasculitis, cryoglobulinemia, and multiple sclerosis, viral or influenza-induced inflammation, or edema.

The above examples are given to illustrate the present invention. It should be understood, however, that the spirit and scope of the invention is not to be limited to the specific conditions or details described in these examples. All publicly available documents referenced herein, including but not limited to U.S. patents, are specifically incorporated by reference.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

1. A compound represented by formula (I): 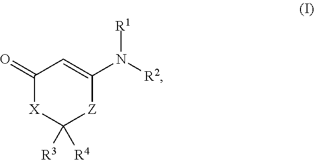
or a pharmaceutically acceptable salt, ester, hydrate thereof, wherein:
X and Z are independently C₀₋₂-alkyl, —(CH₂)ₛ—NH—(CH₂)ₜ—, —(CH₂)ₛ—O—(CH₂)ₜ—, or —(CH₂)ₛ—C(NH₂)—(CH₂)ₜ—, wherein s and t are independently an integer of 0 or 1;
R¹ is selected from the group consisting of H, optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl;
R² is selected from the group consisting of optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl;
R³ is selected from the group consisting of H, optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl; and
R⁴ is selected from the group consisting of optionally-substituted alkyl, alkenyl or alkynyl, optionally-substituted alkoxy, optionally-substituted aryl, optionally-substituted cycloalkyl, optionally-substituted heterocyclic, a substituted alkoxy, optionally-substituted aryl, optionally-substituted cycloalkyl, optionally-substituted heterocyclic, and optionally-substituted heteroalkyl,
with the proviso that when X and Z are CH₂, and R¹ is H, R² is not an unsubstituted C₁-C₈ alkyl or an unsubstituted cyclohexyl or an unsubstituted C₁-C₂ alkyl-phenyl.

2. The compound of claim 1, wherein the compound is selected from a compound of formula (I) or a pharmaceutically acceptable salt, ester, hydrate or prodrug thereof.

3. The compound of claim 1, wherein the compound is selected from a pharmaceutically acceptable salt of compound of formula (I).

4. The compound of claim 1, wherein R² is selected from the group consisting of C₁₋₁₆ alkyl, C₁₋₁₆ alkenyl, and C₁₋₁₆ alkynyl.

5. The compound of claim 1, wherein R² is selected from the group consisting of C₃₋₁₂ cycloalkyl, C₃₋₁₂ cycloalkyl-C₁₋₆alkyl, C₃₋₁₂ heterocycloalkyl, and C₃₋₁₂ heterocycloalkyl-C₁₋₆ alkyl.

6. The compound of claim 1, wherein R² is substituted with at least one of halogen, C₁₋₁₆ alkyl, C₁₋₁₆haloalkyl, hydroxyl, C₁₋₁₆ alkoxy, C₁₋₁₆ haloalkoxy, amino, C₁₋₁₆alkylamino, or di-C₁₋₁₆alkylamino.

7. The compound of claim 1, wherein R⁴ is selected from the group consisting of 6-membered cycloalkyl, 6-membered heterocycloalkyl, 5-membered cycloalkyl, and 5-membered heterocycloalkyl.

8. The compound of claim 1, wherein R⁴ is selected from the group consisting of pyrimidine and pyridine.

9. The compound of claim 1, wherein R⁴ is substituted with at least one substituent selected from the group consisting of halogen, C₁₋₆ alkyl, C₁₋₆ haloalkyl, hydroxyl, C₁₋₆ alkoxy, C₁₋₆haloalkoxy, amino, C₁₋₆ alkylamino, and di-C₁₋₆ alkylamino.

10. The compound of claim 1, wherein R⁴ is selected from the group consisting of:
C₁₋₁₈ alkyl, alkenyl or alkynyl;
C₀₋₁₈ alkyl, alkenyl or alkynyl-C₆₋₁₄ aryl;
C₀₋₁₈ alkyl, alkenyl or alkynyl-C₃₋₁₂ cycloalkyl;
C₀₋₁₈ alkyl, alkenyl or alkynyl-C₃₋₁₂ cycloalkyl-C₆₋₁₄ aryl;
C₀₋₁₈ alkyl, alkenyl or alkynyl-di-C₆₋₁₄ aryl;
C₀₋₁₈ alkyl, alkenyl or alkynyl-C₆₋₁₄ aryl-C₆₋₁₄ aryl;
C₀₋₁₈ alkyl, alkenyl or alkynyl-C₆₋₁₄ aryl-O—C₆₋₁₄ aryl; and
C₀₋₁₈ alkyl, alkenyl or alkynyl-C₆₋₁₄ aryl-C₂₋₅ N or S-heteroaryl.

11. The compound of claim 1, wherein R⁴ is selected from the group consisting of:
C₆₋₁₄ n-alkyl; C₁₋₆ n-alkyl-phenyl, optionally substituted with a phenyl, C₁₋₃-alkyl-phenyl and —O-phenyl.

12. The compound of claim 1, wherein R³ is H.

13. The compound of claim 1, wherein R³ is selected from the group consisting of:
C₁₋₁₈ alkyl, alkenyl or alkynyl;
C₀₋₁₈ alkyl, alkenyl or alkynyl-C₆₋₁₄ aryl;
C₀₋₁₈ alkyl, alkenyl or alkynyl-C₃₋₁₂ cycloalkyl;
C₀₋₁₈ alkyl, alkenyl or alkynyl-C₃₋₁₂ cycloalkyl-C₆₋₁₄ aryl;
C₀₋₁₈ alkyl, alkenyl or alkynyl-di-C₆₋₁₄ aryl;
C₀₋₁₈ alkyl, alkenyl or alkynyl-C₆₋₁₄ aryl-C₆₋₁₄ aryl;
C₀₋₁₈ alkyl, alkenyl or alkynyl-C₆₋₁₄ aryl-O—C₆₋₁₄ aryl; and
C₀₋₁₈ alkyl, alkenyl or alkynyl-C₆₋₁₄ aryl-C₂₋₅ N, O or S-heteroaryl.

14. The compound of claim 1, wherein R¹ is H.

15. The compound of claim 1, wherein R² is selected from the group consisting of:
C₀₋₁₈ alkyl, alkenyl or alkynyl;
C₀₋₁₈ alkyl, alkenyl or alkynyl-C₆₋₁₄ aryl;
C₀₋₁₈ alkyl, alkenyl or alkynyl-C₃₋₁₂ cycloalkyl;
C₀₋₁₈ alkyl, alkenyl or alkynyl-C₃₋₁₂ cycloalkyl-C₆₋₁₄ aryl;
C₀₋₁₈ alkyl, alkenyl or alkynyl-di-C₆₋₁₄ aryl;
C₀₋₁₈ alkyl, alkenyl or alkynyl-C₆₋₁₄ aryl-C₆₋₁₄ aryl;
C₀₋₁₈ alkyl, alkenyl or alkynyl-C₆₋₁₄ aryl-O—C₆₋₁₄ aryl; and
C₀₋₁₈ alkyl, alkenyl or alkynyl-C₆₋₁₄ aryl-C₂₋₅ N, O or S-heteroaryl.

16. The compound of claim 1, wherein X and Z are CH₂.

17. A compound selected from the following:

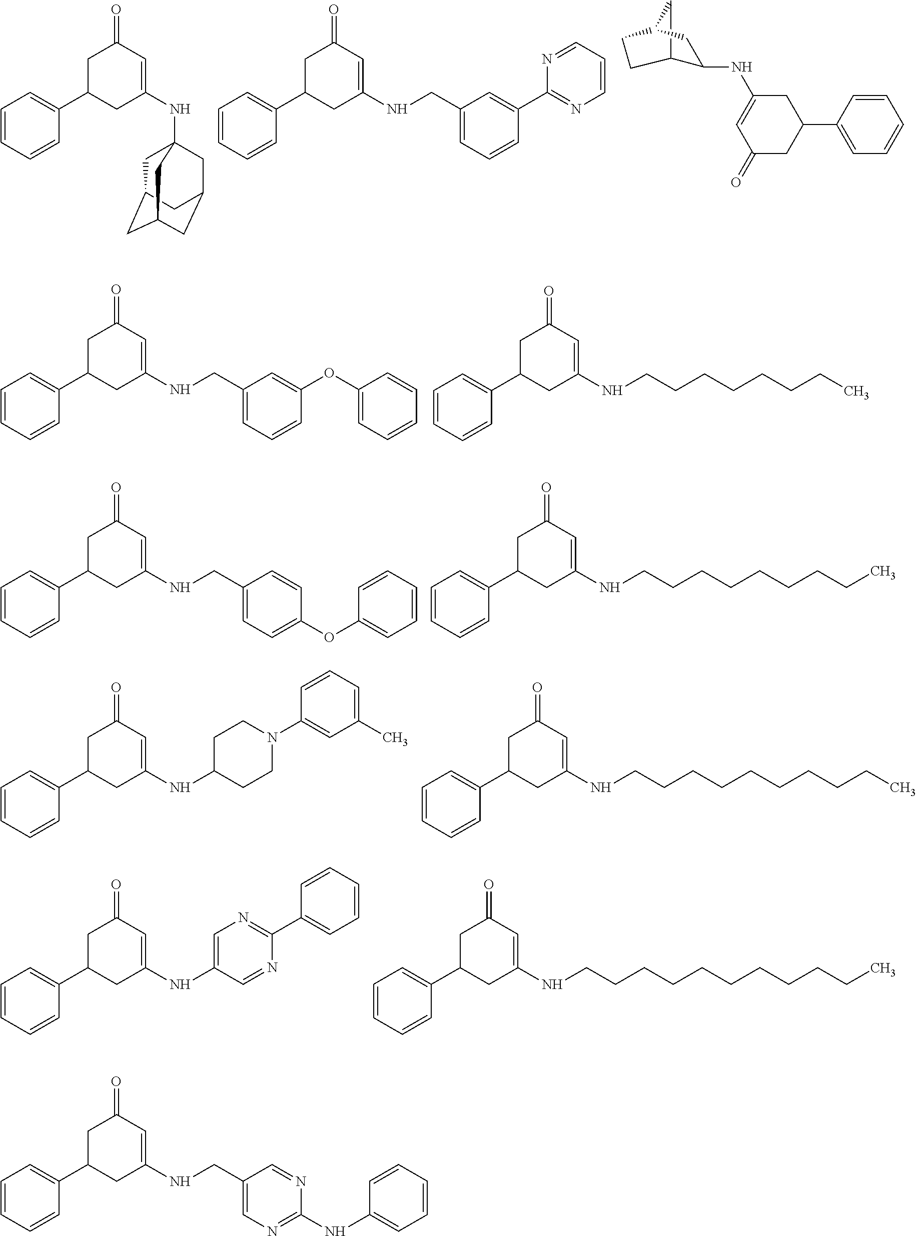

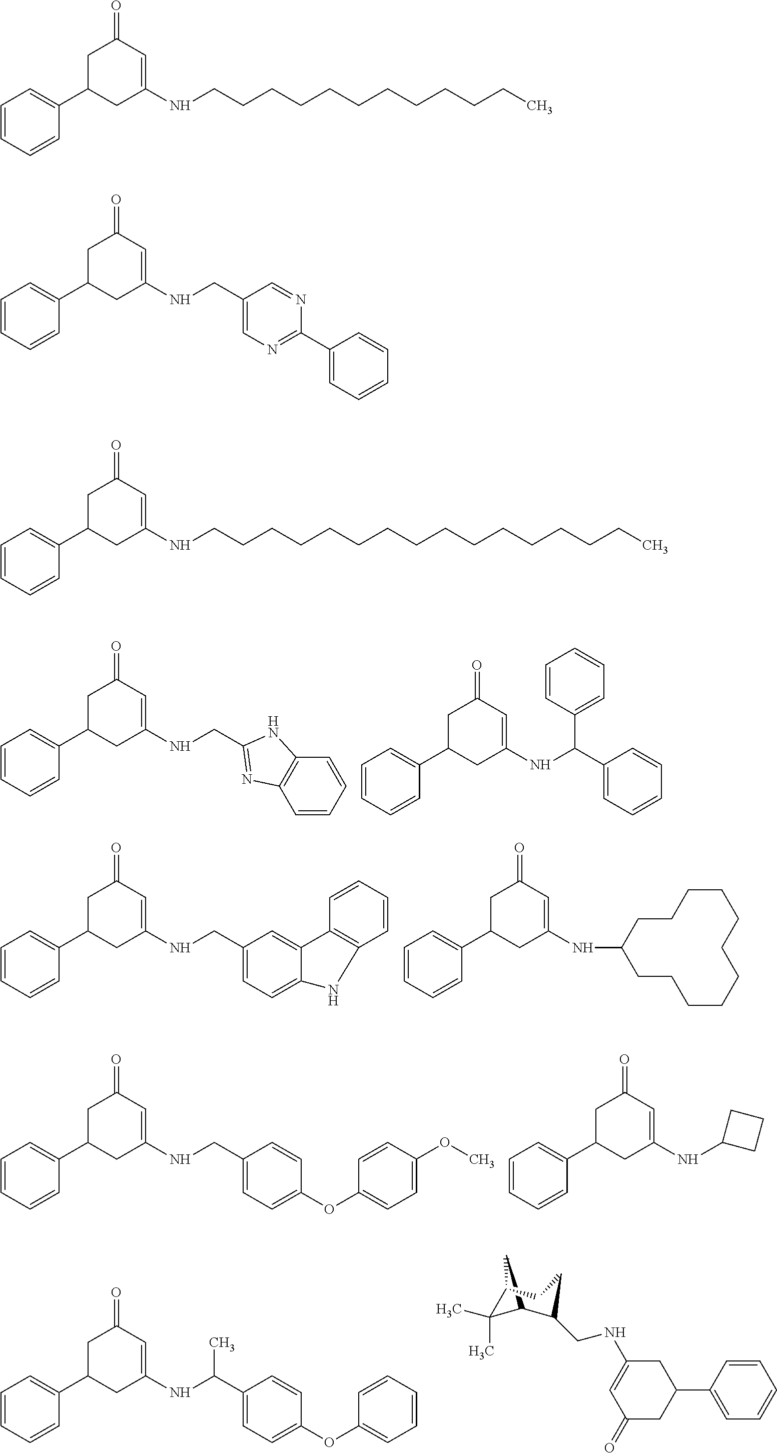

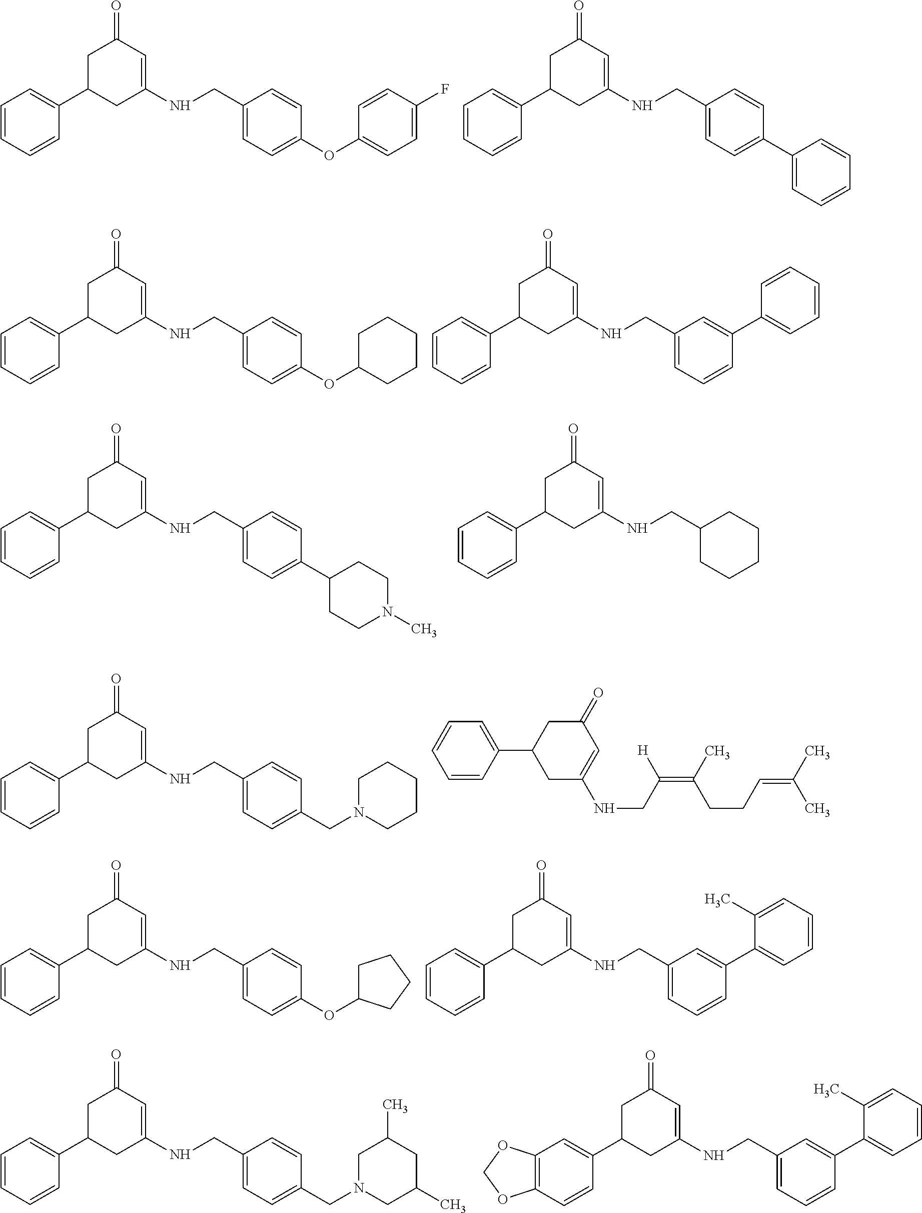

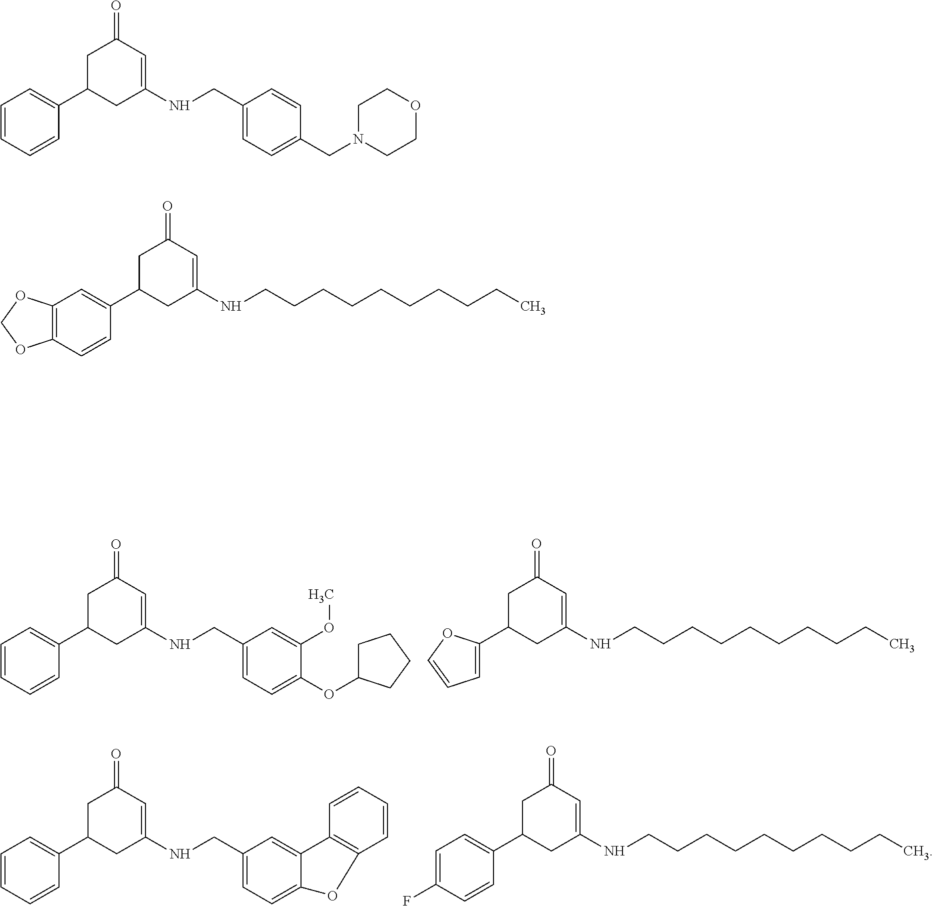

18. A pharmaceutical composition comprising a compound of claim 1, and a pharmaceutically acceptable excipient.

19. A method of treating cancer in a patient in need thereof, the method comprising administering to the patient a composition comprising an effective amount of a compound according to claim 1
wherein the cancer is selected from brain cancer, throat cancer, thyroid cancer, esophagus cancer, tonsil cancer, lung cancer, prostate cancer, colorectal cancer, stomach cancer, liver cancer, pancreatic cancer, gallbladder cancer, bladder cancer, rectal cancer, testicle cancer, breast cancer, cervical cancer, ovarian cancer, skin cancer, melanoma, leukemia, lymphoma, and multiple myeloma.

20. A method of treating an acute and chronic inflammation disorder in a patient in need thereof, the method comprising administering to the patient a composition comprising an effective amount of a compound according to claim 1.

21. The method of claim 20, wherein the acute and chronic inflammation disorder is selected from the group consisting of asthma, chronic obstructive lung disease, pulmonary fibrosis, pneumonitis (including hypersensitivity pneumonitis and radiation pneumonitis), pneumonia, cystic fibrosis, psoriasis, arthritis/rheumatoid arthritis, rhinitis, pharyngitis, cystitis, prostatitis, dermatitis, allergy including hayfever, nephritis, conjunctivitis, encephalitis, meningitis, opthalmitis, uveitis, pleuritis, pericarditis, myocarditis, atherosclerosis, human immunodeficiency virus related inflammation, diabetes, osteoarthritis, psoriatic arthritis, inflammatory bowel disease (Crohn's disease, ulcerative colitis)/colitis, sepsis, vasculitis, bursitis, connective tissue disease, autoimmune diseases such as systemic lupus erythematosis (SLE), polymyalgia rheumatica, scleroderma, Wegener's granulomatosis, temporal arteritis, vasculitis, cryoglobulinemia, and multiple sclerosis, viral or influenza-induced inflammation, and edema.

22. The method of claim 20, wherein the effective amount is from about 0.5 mg to about 500 mg of the compound of formula (I).

23. The method of claim 19, wherein the effective amount is about 0.5 mg/kg to about 500 mg/kg of the compound of formula (I).

24. The method of claim 19, wherein the administration is oral administration, administration via implants, parenteral injection, intravenous injection, intraperitoneal injection, subcutaneous injection, bolus injection, infusion, rectal administration, vaginal administration, transdermal administration, inhalation, or any combination thereof.

25. The method of claim 19, wherein the method further includes a step of administrating at least one anti-inflammatory agent, antimicrobial agent, matrix metalloprotease inhibitor, lipoxygenase inhibitor, cytokine antagonist, immunosuppressant, anti-cancer agent, anti-viral agent, cytokine, growth factor, immunomodulator, prostaglandin, anti-vascular hyperproliferation compound, and combinations thereof either concurrently with the compound formula (I) or in the same course of treatment.

26. The method of claim 19, wherein the method is for treating a condition selected from the group consisting of sepsis, pneumonia, influenza-induced inflammation, edema, neuropathy, colitis, arthritis, Crohn s disease, diabetes, skin, eye and ear inflammation (e.g., psoriasis, uveitis/opthalmitis, external otitis), systemic lupus erythematosis (SLE), or systemic lupus erythematosis (SLE) in the patient in need thereof.

27.-28. (canceled)
